SEQUENCE LISTING (280 sequences)

SEQ ID NO: 1-100 - skipped (100 entries)

SEQ ID NO: 101 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 102 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 103 - protein, 233 aa, Homo sapiens
MWQLLLPTALLLLVSAGMRTEDLPKAVVFLEPQWYRVLEKDSVTLKCQGAYSPEDNSTQWFHNENLISSQASSYFIDAATVDDSGEYRCQTNLSTLSDPVQLEVHVGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 104 - protein, 233 aa, Homo sapiens
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 105 - protein, 233 aa, Homo sapiens
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 106 - protein, 254 aa, Macaca fascicularis
MWQLLLPTALLLLVSAGMRAEDLPKAVVFLEPQWYRVLEKDRVTLKCQGAYSPEDNSTRWFHNESLISSQTSSYFIAAARVNNSGEYRCQTSLSTLSDPVQLEVHIGWLLLQAPRWVFKEEESIHLRCHSWKNTLLHKVTYLQNGKGRKYFHQNSDFYIPKATLKDSGSYFCRGLIGSKNVSSETVNITITQDLAVSSISSFFPPGYQVSFCLVMVLLFAVDTGLYFSMKKSIPSSTRDWEDHKFKWSKDPQDK

SEQ ID NO: 107 - protein, 231 aa, Homo sapiens
EPKSSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 241 aa, synthetic construct
  REGION 1..241: note = Synthetic
EVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGGGSGGGGSGGGGSQPVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 109 - protein, 5 aa, synthetic construct
  REGION 1..5: note = Synthetic
SYAVG

SEQ ID NO: 110 - protein, 17 aa, synthetic construct
  REGION 1..17: note = Synthetic
AIRRSDGTTDYVDSVKG

SEQ ID NO: 111 - protein, 11 aa, synthetic construct
  REGION 1..11: note = Synthetic
SDSDYEPLGDY

SEQ ID NO: 112 - protein, 120 aa, synthetic construct
  REGION 1..120: note = Synthetic
QVQLVESGGGVVQPGGSLTLSCEASGRTFGSYAVGWFRQALGRGREFVAAIRRSDGTTDYVDSVKGRFTISRDNAKNTVYLQMNSLKPEDTAVYYCAASDSDYEPLGDYWGQGTQVTVSS

SEQ ID NO: 113 - DNA, 360 bp, synthetic construct
  misc_feature 1..360: note = Synthetic
  source 1..360: mol_type = other DNA
caggtgcagctcgtggagtctgggggcggcgtggtgcagcctggggggtctctgacactctcctgtgaagcctctggacgcaccttcggcagctatgccgtgggctggttccgccaggctcttgggagggggcgtgagtttgtagcagctattaggcggagtgatggtactacagactatgtagactccgtgaagggccgattcaccatctccagagacaacgccaagaacacggtgtatctgcaaatgaacagcctgaaacctgaggacacggccgtttattactgtgcagcatcggatagcgactatgaacccttaggggactactggggccaggggacccaggtcaccgtctcctca

SEQ ID NO: 114 - protein, 17 aa, synthetic construct
  REGION 1..17: note = Synthetic
AIRRSDATTDYVDSVKG

SEQ ID NO: 115 - protein, 120 aa, synthetic construct
  REGION 1..120: note = Synthetic
EVQLVESGGGLVQPGGSLRLSCAASGRTFGSYAVGWFRQAPGKGREWVAAIRRSDATTDYVDSVKGRFTISRDNAKNSVYLQMNSLRPEDTAVYYCAASDSDYEPLGDYWGQGTLVTVSS

SEQ ID NO: 116 - DNA, 360 bp, synthetic construct
  misc_feature 1..360: note = Synthetic
  source 1..360: mol_type = other DNA
gaggtgcagctcgtggaatccgggggcgggctggtgcaacccggcgggagcctgagactgagctgcgccgctagcggcagaaccttcggcagctacgccgtgggctggttcagacaagcccccggcaagggcagagagtgggtggccgccatcagaagaagcgacgccaccaccgactacgtggacagcgtgaagggcagattcaccatcagcagagacaacgccaagaacagcgtgtacctgcagatgaacagcctgagacccgaggacaccgccgtgtactactgcgccgcctccgacagcgactacgagcctctgggcgattactggggccaaggcaccctggtgaccgtgagcagc

SEQ ID NO: 117 - protein, 120 aa, synthetic construct
  REGION 1..120: note = Synthetic
EVQLVESGGGLVQPGGSLRLSCAASGRTFGSYAVGWFRQAPGRGREFVAAIRRSDGTTDYVDSVKGRFTISRDNAKNTVYLQMNSLKPEDTAVYYCAASDSDYEPLGDYWGQGTLVTVSS

SEQ ID NO: 118 - DNA, 360 bp, synthetic construct
  misc_feature 1..360: note = Synthetic
  source 1..360: mol_type = other DNA
gaggtgcagctcgtggaatccgggggcgggctggtgcaacccggcgggagcctgagactgagctgcgccgctagcggcagaaccttcggcagctacgccgtgggctggttcagacaagcccccggcagaggcagagagttcgtggccgccatcagaagaagcgacggcaccaccgactacgtggacagcgtgaagggcagattcaccatcagcagagacaacgccaagaacaccgtgtacctgcagatgaacagcctgaagcccgaggacaccgccgtgtactactgcgccgcctccgacagcgactacgagcctctgggcgattactggggccaaggcaccctggtgaccgtgagcagc

SEQ ID NO: 119 - protein, 120 aa, synthetic construct
  REGION 1..120: note = Synthetic
EVQLVESGGGLVQPGGSLRLSCAASGRTFGSYAVGWFRQAPGKGREFVAAIRRSDATTDYVDSVKGRFTISRDNAKNTVYLQMNSLKPEDTAVYYCAASDSDYEPLGDYWGQGTLVTVSS

SEQ ID NO: 120 - DNA, 360 bp, synthetic construct
  misc_feature 1..360: note = Synthetic
  source 1..360: mol_type = other DNA
gaggtgcagctcgtggaatccgggggcgggctggtgcaacccggcgggagcctgagactgagctgcgccgctagcggcagaaccttcggcagctacgccgtgggctggttcagacaagcccccggcaagggcagagagttcgtggccgccatcagaagaagcgacgccaccaccgactacgtggacagcgtgaagggcagattcaccatcagcagagacaacgccaagaacaccgtgtacctgcagatgaacagcctgaagcccgaggacaccgccgtgtactactgcgccgcctccgacagcgactacgagcctctgggcgattactggggccaaggcaccctggtgaccgtgagcagc

SEQ ID NO: 121 - protein, 449 aa, synthetic construct
  REGION 1..449: note = Synthetic
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 216 aa, synthetic construct
  REGION 1..216: note = Synthetic
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123-243 - skipped (121 entries)

SEQ ID NO: 244 - protein, 4 aa, synthetic construct
  REGION 1..4: note = Synthetic
GGGS

SEQ ID NO: 245 - protein, 4 aa, synthetic construct
  REGION 1..4: note = Synthetic
SGGG

SEQ ID NO: 246 - protein, 5 aa, synthetic construct
  REGION 1..5: note = Synthetic
GGGGS

SEQ ID NO: 247 - protein, 7 aa, synthetic construct
  REGION 1..7: note = Synthetic
GGGGSGS

SEQ ID NO: 248 - protein, 7 aa, synthetic construct
  REGION 1..7: note = Synthetic
GGGGSGS

SEQ ID NO: 249 - protein, 8 aa, synthetic construct
  REGION 1..8: note = Synthetic
GGGGSGGS

SEQ ID NO: 250 - protein, 10 aa, synthetic construct
  REGION 1..10: note = Synthetic
GGGGSGGGGS

SEQ ID NO: 251 - protein, 15 aa, synthetic construct
  REGION 1..15: note = Synthetic
GGGGSGGGGSGGGGS

SEQ ID NO: 252 - protein, 16 aa, synthetic construct
  REGION 1..16: note = Synthetic
AKTTPKLEEGEFSEAR

SEQ ID NO: 253 - protein, 17 aa, synthetic construct
  REGION 1..17: note = Synthetic
AKTTPKLEEGEFSEARV

SEQ ID NO: 254 - protein, 9 aa, synthetic construct
  REGION 1..9: note = Synthetic
AKTTPKLGG

SEQ ID NO: 255 - protein, 10 aa, synthetic construct
  REGION 1..10: note = Synthetic
SAKTTPKLGG

SEQ ID NO: 256 - protein, 17 aa, synthetic construct
  REGION 1..17: note = Synthetic
AKTTPKLEEGEFSEARV

SEQ ID NO: 257 - protein, 6 aa, synthetic construct
  REGION 1..6: note = Synthetic
SAKTTP

SEQ ID NO: 258 - protein, 10 aa, synthetic construct
  REGION 1..10: note = Synthetic
SAKTTPKLGG

SEQ ID NO: 259 - protein, 6 aa, synthetic construct
  REGION 1..6: note = Synthetic
RADAAP

SEQ ID NO: 260 - protein, 9 aa, synthetic construct
  REGION 1..9: note = Synthetic
RADAAPTVS

SEQ ID NO: 261 - protein, 12 aa, synthetic construct
  REGION 1..12: note = Synthetic
RADAAAAGGPGS

SEQ ID NO: 262 - protein, 27 aa, synthetic construct
  REGION 1..27: note = Synthetic
RADAAAAGGGGSGGGGSGGGGSGGGGS

SEQ ID NO: 263 - protein, 6 aa, synthetic construct
  REGION 1..6: note = Synthetic
SAKTTP

SEQ ID NO: 264 - protein, 10 aa, synthetic construct
  REGION 1..10: note = Synthetic
SAKTTPKLGG

SEQ ID NO: 265 - protein, 18 aa, synthetic construct
  REGION 1..18: note = Synthetic
SAKTTPKLEEGEFSEARV

SEQ ID NO: 266 - protein, 5 aa, synthetic construct
  REGION 1..5: note = Synthetic
ADAAP

SEQ ID NO: 267 - protein, 12 aa, synthetic construct
  REGION 1..12: note = Synthetic
ADAAPTVSIFPP

SEQ ID NO: 268 - protein, 5 aa, synthetic construct
  REGION 1..5: note = Synthetic
TVAAP

SEQ ID NO: 269 - protein, 12 aa, synthetic construct
  REGION 1..12: note = Synthetic
TVAAPSVFIFPP

SEQ ID NO: 270 - protein, 6 aa, synthetic construct
  REGION 1..6: note = Synthetic
QPKAAP

SEQ ID NO: 271 - protein, 13 aa, synthetic construct
  REGION 1..13: note = Synthetic
QPKAAPSVTLFPP

SEQ ID NO: 272 - protein, 6 aa, synthetic construct
  REGION 1..6: note = Synthetic
AKTTPP

SEQ ID NO: 273 - protein, 13 aa, synthetic construct
  REGION 1..13: note = Synthetic
AKTTPPSVTPLAP

SEQ ID NO: 274 - protein, 6 aa, synthetic construct
  REGION 1..6: note = Synthetic
AKTTAP

SEQ ID NO: 275 - protein, 13 aa, synthetic construct
  REGION 1..13: note = Synthetic
AKTTAPSVYPLAP

SEQ ID NO: 276 - protein, 6 aa, synthetic construct
  REGION 1..6: note = Synthetic
ASTKGP

SEQ ID NO: 277 - protein, 13 aa, synthetic construct
  REGION 1..13: note = Synthetic
ASTKGPSVFPLAP

SEQ ID NO: 278 - protein, 15 aa, synthetic construct
  REGION 1..15: note = Synthetic
GENKVEYAPALMALS

SEQ ID NO: 279 - protein, 15 aa, synthetic construct
  REGION 1..15: note = Synthetic
GPAKELTPLKEAKVS

SEQ ID NO: 280 - protein, 15 aa, synthetic construct
  REGION 1..15: note = Synthetic
GHEAAAVMQVQYPAS

(19) United States
(12) Patent Application Publication
(10) Pub. No.: US 2026/0008853 A1
(43) Pub. Date: Jan. 8, 2026

(54) CD16A Antibodies and Methods of Use

(71) Applicant: BeOne Medicines I GmbH, Basel (CH)

(72) Inventors: Qiansheng Ren, Beijing (CN); Liang Qu, Beijing (CN); Wenjie Wang, Beijing (CN); Liu Xue, Beijing (CN); Zhuo Li, Shanghai (CN); Chichi Huang, Cambridge, MA (US)

(73) Assignee: BeOne Medicines I GmbH, Basel (CH)

(21) Appl. No.: 19/315,937

(22) Filed: Sep. 2, 2025

Related U.S. Application Data
(63) Continuation of application No. PCT/CN2024/079594, filed on Mar. 1, 2024.

(30) Foreign Application Priority Data
Mar. 3, 2023 (WO) PCT/CN2023/079509

(51) Int. Cl. C07K 16/28

(52) U.S. Cl. CPC C07K 16/283; C07K 2317/24; C07K 2317/31; C07K 2317/33; C07K 2317/565; C07K 2317/569; C07K 2317/92; C07K 2317/94

(57) ABSTRACT

The present disclosure provides antibodies and antigen-binding fragments that specifically bind to human CD16A.

### CROSS-REFERENCE TO RELATED APPLICATION

This application is a continuation of PCT Application No. PCT/CN2024/079594, filed Mar. 1, 2024, entitled “CD16A Antibodies and Methods of Use,” which claims the benefit of priority of PCT Application No. PCT/CN2023/079509, filed Mar. 3, 2023, entitled “CD16A Antibodies and Methods of Use,” which is hereby incorporated by reference in its entirety.

### SEQUENCE LISTING

The present application is filed with a Sequence Listing in electronic format. The Sequence Listing is provided as a file entitled “01368-0055-00PCT_SL.xml” created on Feb. 27, 2024, which is 119,384 bytes in size. The information in the electronic format of the sequence listing is incorporated herein by reference in its entirety.

### FIELD OF THE DISCLOSURE

Disclosed herein are antibodies that specifically bind to human CD16A with selectivity over human CD16B.

### BACKGROUND

Natural Killer (NK) cells are critical innate immune lymphocytes that mediate anti-viral and anti-tumor responses. Monoclonal antibodies (mAbs) targeting tumors could exert their antibody dependent cell mediated cytotoxicity (ADCC) function through binding to the low affinity Fc receptor, CD16A, on the surface of NK cells.

CD16A (FCGRIIIA, FCGR3A) is a type I membrane protein with two Ig-like domains that have low affinity for IgG. CD16A is found widely in myeloid cells, such as macrophages, dendritic cells, mast cells, and eosinophils as well as in NK cells and T cells, but not in neutrophils. When bound by antigen-IgG complexes, CD16A can associate with an adaptor protein FcR gamma chain (FcγRI) to mediate signal transduction through its intracellular immunoreceptor tyrosine-based activation motif (ITAM) and activate INK cells for cytolysis.

Genetic polymorphism in CD16A exists at two sites: 48(L/R/H) and 158(F/V). The presence of a valine (V/V or V/F) at site 158 in CD16A has been shown to enhance NK cell binding to IgG1 or IgG3 and produce a higher level of NK cell mediated ADCC when compared with CD16A (F/F 158). Several clinical reports have shown an improved progression-free survival for patients with CD16A (V/V 158) versus patients with CD16A (F/F 158) when treated with various monoclonal antibodies. These studies highlighted the importance of CD16A polymorphism for NK cell effector function and that manipulation of an NK cell engager could regulate its cytotoxic activity.

Another CD16 isoform, CD16B (FCGR3B, FCGRIIIB), exists in humans but not in mouse, rat, rabbit, llama, or cyno monkey. CD16B is highly homologous to CD16A and can bind to IgG at a low affinity range. CD16B is a GPI-anchored protein selectively expressed in neutrophils and eosinophils. It is generally believed that CD16B functions as a decoy receptor that can bind to IgG complexes without triggering activation. Although it is still unclear whether CD16B engagement on neutrophils will be clinically important, it is desirable to spare CD16B engagement at least as a drug sink in neutrophils.

Although therapeutic antibodies targeting tumors are successful in cancer treatment, they are not without limitations, such as off-target binding to other Fc receptors and competition with serum IgG for CD16A binding. Alternative methods to enhance NK cell based ADCC functions include antibodies with enhanced Fc binding activities through Fc engineering or glycoengineering, and bi- or tri-specific NK cell engagers (NKCE). These NKCEs are designed with one arm specifically recruiting NK cells and another arm binding to tumor cells. When compared with mono-specific antibodies, NKCEs are more flexible with variable binding affinities, valences, and targets. Thus, they may provide unique opportunities for cancer treatment. Indeed, several NKCE molecules that entered clinical or preclinical phases have been reported. An anti-human CD16A specific single chain Fv (scFv) was initially identified from a naïve phage library and underwent affinity maturation. When paired with disclosed tumor associated antigens (TAAs) such as CD30, EGFR, BCMA, or CD123, these NKCEs engaging NK cells through CD16A have already demonstrated their superior efficacy versus antibodies in in vitro and in vivo assays.

There is a need for anti-CD16A antibodies that could specifically bind to human CD16A with minimal or no binding to human CD16B, and that are useful as a NKCE to pair with a TAA to treat TAA-positive tumors.

### SUMMARY OF THE DISCLOSURE

The present disclosure is directed to anti-CD16A antibodies and antigen-binding fragments thereof that specifically bind CD16A (including CD16A 158F and 158V). The antibodies and antigen-binding fragments are selective for CD16A over CD16B. The antibodies may activate NK cells upon binding to CD16A.

In embodiments, the present disclosure is directed to an anti-CD16A antibody or antigen-binding fragment thereof that specifically binds to human CD16A.

In embodiments, the anti-CD16A antibody or antigen-binding fragment thereof has selectivity for human CD16A over human CD16B.

In embodiments, the anti-CD16A antibody or antigen-binding fragment has cross-binding affinity to both human and cyno CD16A.

In embodiments, the anti-CD16A antibody or antigen-binding fragment thereof is a monoclonal antibody, a chimeric antibody, a humanized antibody, a human engineered antibody, a single chain antibody (scFv), a Fab fragment, a F(ab′)₂ fragment, a heavy chain antibody (HcAb), or a VHH.

In embodiments, the anti-CD16A antibody or antigen-binding fragment thereof is a VHH.

In embodiments, the anti-CD16A antibody or antigen-binding fragment thereof comprises at least one heavy chain CDR selected from the group consisting of:
a) a heavy chain CDR1 sequence of SEQ ID NO: 109;
b) a heavy chain CDR2 sequence selected from the group consisting of SEQ ID NO: 110 and 114; and
c) a heavy chain CDR3 sequence of SEQ ID NO: 111.

In embodiments, the anti-CD16A antibody or antigen-binding fragment thereof comprises each of the following heavy chain CDRs:
a) a heavy chain CDR1 sequence of SEQ ID NO: 109;
b) a heavy chain CDR2 sequence selected from the group consisting of SEQ ID NO: 110 and 114; and
c) a heavy chain CDR3 sequence of SEQ ID NO: 111.

In embodiments, the anti-CD16A antibody or antigen-binding fragment comprises a heavy chain variable region that comprises:
a) a HCDR1 of SEQ ID NO: 109, (b) a HCDR2 of SEQ ID NO: 110, and (c) a HCDR3 of SEQ ID NO: 111; or
b) a HCDR1 of SEQ ID NO: 109, (b) a HCDR2 of SEQ ID NO: 114, and (c) a HCDR3 of SEQ ID NO: 111.

In embodiments, the anti-CD16A antibody or antigen-binding fragment thereof comprises a heavy chain variable region (VH) comprising at least one amino acid sequence at least 90, 91, 92, 93, 94, 95, 96, 97, 98, or 99% identical to SEQ ID NO: 112, 115, 117, or 119.

In embodiments, the anti-CD16A antibody or antigen-binding fragment thereof comprises a heavy chain variable region (VH) comprising at least one amino acid sequence selected from SEQ ID NO: 112, 115, 117, and 119 with one, two, three, four, five, six, seven, eight, nine, or ten amino acid deletions, additions, or substitutions within at least one of SEQ ID NO: 112, 115, 117, and 119.

In embodiments, the anti-CD16A antibody or antigen-binding fragment thereof comprises at least one amino acid sequence selected from the group consisting of SEQ ID NO: 112, 115, 117, and 119.

The present disclosure is also directed to multi-specific antibodies or antigen-binding fragments thereof comprising at least a first antigen-binding domain that specifically binds human CD16A, wherein said first antigen-binding domain comprises an antibody-binding fragment that specifically binds to human CD16A.

In embodiments, in the multi-specific antibody or antigen-binding fragment, said first antigen-binding domain has selectivity for human CD16A over human CD16B.

In embodiments, in the multi-specific antibody or antigen-binding fragment, said first antigen-binding domain has cross-binding affinity to both human and cyno CD16A.

In embodiments, in the multi-specific antibody or antigen-binding fragment, said first antigen-binding domain is an VHH.

In embodiments, the multi-specific antibody or antigen-binding fragment is a monoclonal anti body, a chimeric antibody, a humanized antibody, a human engineered antibody, a single chain antibody (scFv), a Fab fragment, a F(ab′)₂ fragment, a heavy chain antibody (HcAb), and a VHH.

In embodiments, the multi-specific antibody or antigen-binding fragment comprises at least one heavy chain CDR selected from the group consisting of:
a) a heavy chain CDR1 sequence of SEQ ID NO: 109;
b) a heavy chain CDR2 sequence selected from the group consisting of SEQ ID NO: 110 and 114; and
c) a heavy chain CDR3 sequence of SEQ ID NO: 111.

In embodiments, the multi-specific antibody or antigen-binding fragment comprises each of the following heavy chain CDRs:
a) a heavy chain CDR1 sequence of SEQ ID NO: 109;
b) a heavy chain CDR2 sequence selected from the group consisting of SEQ ID NO: 110 and 114; and
c) a heavy chain CDR3 sequence of SEQ ID NO: 111.

In embodiments, the multi-specific antibody or antigen-binding fragment comprises a heavy chain variable region that comprises:
a) a HCDR1 of SEQ ID NO: 109, (b) a HCDR2 of SEQ ID NO: 110, and (c) a HCDR3 of SEQ ID NO: 111; or
b) a HCDR1 of SEQ ID NO: 109, (b) a HCDR2 of SEQ ID NO: 114, and (c) a HCDR3 of SEQ ID NO: 111.

In embodiments, the multi-specific antibody or antigen-binding fragment comprises a heavy chain variable region (VH) comprising at least one amino acid sequence at least 90, 91, 92, 93, 94, 95, 96, 97, 98, or 99% identical to SEQ ID NO: 112, 115, 117, or 119.

In embodiments, the multi-specific antibody or antigen-binding fragment comprises a heavy chain variable region (VH) comprising at least one amino acid sequence selected from SEQ ID NO: 112, 115, 117, or 119 with one, two, three, four, five, six, seven, eight, nine, or ten amino acid deletions, additions, or substitutions within at least one of SEQ ID NO: 112, 115, 117, or 119.

In embodiments, the multi-specific antibody or antigen-binding fragment comprises at least one amino acid sequence selected from the group consisting of SEQ ID NO: 112, 115, 117, and 119.

The present disclosure is also directed to a VHH, wherein said VHH specifically binds to human CD16A.

In embodiments, the VHH has selectivity for human CD16A over human CD16B.

In embodiments, the VHH has cross-binding affinity to both human and cyno CD16A.

In embodiments, the VHH is a humanized antibody or a human engineered antibody.

In embodiments, the VHH comprises at least one heavy chain CDR selected from the group consisting of:
a) a heavy chain CDR1 sequence of SEQ ID NO: 109;
b) a heavy chain CDR2 sequence selected from the group consisting of SEQ ID NO: 110 and 114; and
c) a heavy chain CDR3 sequence of SEQ ID NO: 111.

In embodiments, the VHH comprises each of the following heavy chain CDRs:
a) a heavy chain CDR1 sequence of SEQ ID NO: 109;
b) a heavy chain CDR2 sequence selected from the group consisting of SEQ ID NO: 110 and 114; and
c) a heavy chain CDR3 sequence of SEQ ID NO: 111.

In embodiments, the VHH comprises a heavy chain variable region that comprises:
a) a HCDR1 of SEQ ID NO: 109, (b) a HCDR2 of SEQ ID NO: 110, and (c) a HCDR3 of SEQ ID NO: 111; or
b) a HCDR1 of SEQ ID NO: 109, (b) a HCDR2 of SEQ ID NO: 114, and (c) a HCDR3 of SEQ ID NO: 111.

In embodiments, the VHH comprises a heavy chain variable region (VH) comprising at least one amino acid sequence at least 90, 91, 92, 93, 94, 95, 96, 97, 98, or 99% identical to SEQ ID NO: 112, 115, 117, or 119.

In embodiments, the VHH comprises a heavy chain variable region (VH) of at least one amino acid sequence selected from SEQ ID NO: 112, 115, 117, and 119 with one, two, three, four, five, six, seven, eight, nine, or ten amino acid insertions, deletions, or substitutions within at least one of SEQ ID NO: 112, 115, 117, or 119.

In embodiments, the VHH comprises at least one amino acid sequence selected from the group consisting of SEQ ID NO: 112, 115, 117, and 119.

The present disclosure also provides a construct, which comprises an antibody or antigen-binding fragment disclosed herein.

In embodiments, the construct comprises one or more VHH disclosed herein.

In embodiments, the construct is a multi-specific antibody, a heavy chain antibody, a bivalent VHH, a biparatopic VHH, a bispecific VHH, a VHH-scFv, a VHH-cytokine, a VHH-drug, a VHH-nanoparticles, a VHH-virus, or a VHH-imaging probe.

In embodiments, the present disclosure provides anti-CD16A antibodies, antigen-binding fragments thereof, or constructs that specifically bind and have high affinity for human CD16A, show superior overall biophysical properties, and/or show superior pharmacokinetics. The superior biophysical properties may be Tm and/or Tagg.

In embodiments, the anti-CD16A antibodies, antigen-binding fragments thereof, or constructs have at least one of the following features:
(1) specific binding and high affinity for human CD16A;
(2) superior pharmacokinetics;
(3) superior overall biophysical properties, e.g., Tm or Tagg; or
(4) being a humanized antibody having low immunogenicity risk to humans.

In embodiments, the present disclosure provides anti-CD16A antibodies, antigen-binding fragments thereof, or constructs that are humanized antibodies having low immunogenicity risk to human, while maintaining specific binding and high affinity to human CD16A, and showing superior overall biophysical properties.

The present disclosure is also directed to a pharmaceutical composition comprising any of the anti-CD16A antibodies, antigen-binding fragments, or construct disclosed herein and a pharmaceutically acceptable carrier.

The present disclosure also provides an isolated nucleic acid that encodes the anti-CD16A antibodies or antigen-binding fragments disclosed herein.

The present disclosure also provides a vector comprising the nucleic acids disclosed herein.

The present disclosure also provides a host cell comprising the nucleic acids or the vectors disclosed herein.

The present disclosure also provides a process for producing an anti-CD16A antibody or antigen-binding fragment thereof comprising cultivating the host cells disclosed herein and recovering the antibody or antigen-binding fragment from the culture.

### BRIEF DESCRIPTION OF THE DRAWINGS

FIG. 1 is a bar graph showing the ELISA analysis results of a representative top clone BG523P vs LS21.

FIGS. 2A-2C are line graphs showing the FACS analysis of a representative top clone, BG523P, vs LS21 in NK92mi/CD16A (V158) (FIG. 2A), NK92mi/CD16B (NA1) (FIG. 2B), and NK92mi/CD16B (NA2) (FIG. 2C) cells.

FIGS. 3A-3B are lines graphs showing the FACS binding comparison of BG523P and BG524P (FIG. 3A) or BG523P, BG525P, and BG526P (FIG. 3B) to CD16A 158F overexpressing cells (NK92mi/CD16A 158F cell line).

FIG. 4 is a bar graph showing the FACS binding signals of BG523P, BG525P, and BG526P at 300 nM to CD16B overexpressing cells (NK92mi/CD16B NA1 and NK92mi/CD16B NA2 cell lines).

FIGS. 5A-5C are line graphs showing FACS-based human IgG competition for NK92mi/CD16A 158F binding for BG523P and its humanized VHHs (BG525P and BG526P) in the presence or absence of 10 mg/mL recombinant CB6 human IgG1. FIG. 5A shows the IgG competition effect on BG523P binding to NK92mi/CD16A 158F; FIG. 5B shows the IgG competition effect on BG525P binding to NK92mi/CD16A 158F; and FIG. 5C shows the IgG competition effect on BG526P binding to NK92mi/CD16A 158F.

### DEFINITIONS

Unless specifically defined below or elsewhere in this document, all other technical and scientific terms used herein have the meaning commonly understood by one of ordinary skill in the art.

As used herein, including in the appended claims, the singular forms of words such as “a”, “an,” and “the” include their corresponding plural references unless the context clearly dictates otherwise.

The term “of” is used to mean, and is used interchangeably with, the term “and/or” unless the context clearly dictates otherwise.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within one or more than one standard deviation per the practice in the art. “About” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” should be assumed to be within an acceptable error range for that particular value or composition.

The term “CD16A” refers to a type I membrane protein with two Ig-like domains that have low affinity for IgG, and is also known as FCGRIIIA and FCGR3A. The amino acid sequence of human CD16A (P08637) can be found with Uniprot P08637 in Uniprot Database.

The terms “administration,” and “administering,” as used herein, when applied to an animal, human, subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell.

The term “subject” or “patient” herein includes any organism, preferably an animal, more preferably a mammal (e.g., rat, mouse, dog, cat, rabbit, primate) and most preferably a human (e.g., a patient comprising, or at risk of having, a disorder described herein).

“Treating” any disease or disorder refers in one aspect to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically (e.g., stabilization of a discernible symptom), physiologically (e.g., stabilization of a physical parameter), or both.

The term “affinity” as used herein refers to the strength of interaction between antibody and antigen. Within the antigen, the variable regions of the antibody interact through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997); Lefranc, M.-P., The Immunologist, 7, 132-136 (1999); Lefranc, M.-P. et al., Dev. Comp. Immunol., 27, 55-77 (2003)).

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA, and IgY), or subclass (e.g., IgG1, IgG2, IgG3, IgG4, IgA1, and IgA2).

The term “chimeric antibody” means molecules made up of domains from different species, i.e., fusing the variable domain of an antibody from one host species (e.g. mouse, rabbit, llama, etc.) with the constant domain of an antibody from a different species (e.g. human).

The term “monoclonal antibody” or “mAb” or “Mab” herein means a population of substantially homogeneous antibodies, i.e., the antibody molecules in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively.

Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (FR3), CDR-3 (CDR3), and FR-4 (FR4). The positions of the CDRs and framework regions can be determined using various well known definitions in the art, e.g., Kabat, Chothia, AbM, and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mot. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2), and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2, and LCDR3 in the light chain variable domain and HCDR1, HCDR2, and HCDR3 in the heavy chain variable domain). See, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but are not limited to, Fab, Fab′, F(ab′)2, and Fv fragments; diabodies; linear antibodies; single-chain antibody molecules, e.g., single chain Fv (ScFv); nanobodies (or VHH antibody); multi-specific antibodies formed from antibody fragments; and bicyclic peptides (Hurov, K. et al., 2021. Journal for ImmunoTherapy of Cancer, 9(11)).

As used herein, an antibody or antigen-binding antibody fragment “specifically binds” to an antigen (e.g., a protein), meaning the antibody exhibits preferential binding to that target as compared to other proteins, but this specificity does not require absolute binding specificity. A “specific” or “selective” binding reaction is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten times when compared to the background level of binding and does not specifically bind in a significant amount to other antigens present in the sample.

“Antigen-binding domain” as used herein, comprises at least six CDRs (or three CDRs in terms of single domain antibody) and specifically binds to an epitope. An “antigen-binding domain” of a multi-specific antibody (e.g., a bispecific antibody) comprises a first antigen binding domain that specifically binds to a first epitope and a second antigen binding domain also comprised of at least three CDRs that specifically binds to a second epitope. Multi-specific antibodies can be bispecific, trispecific, tetraspecific, etc., with antigen binding domains directed to each specific epitope. Multi-specific antibodies can be multivalent (e.g., a bispecific tetravalent antibody) that comprises multiple antigen binding domains, for example, 2, 3, 4, or more antigen binding domains that specifically bind to a first epitope and 2, 3, 4, or more antigen binding domains that specifically bind a second epitope. An “antigen-binding domain” of a single chain antibody, e.g., a heavy chain antibody, or a VHH, comprises an antigen binding domain that specifically binds to an epitope without pairing with an additional variable domain. The binding site of an immunoglobulin single variable domain is formed by a single VH/VHH or VL domain.

The terms “VHH domain,” “VHH antibody,” “VHH antibody fragment,” and “VHH” (also known as a nanobody), as used herein, refer to that which was originally described as the antigen binding domain of heavy chain-only antibodies produced by camelids (Naturally occurring antibodies devoid of light chains, C. Hamers-Casterman et al., Nature, volume 363, pages 446-448 (1993)) and is distinguished from the heavy chain variable domains (VH domain) of a conventional tetramer antibody. The VHH antibody retains the immunoglobulin fold of conventional four-chain antibodies, with only three hypervariable loops—CDR1, CDR2, and CDR3—to bind to its target. Many VHHs bind to their targets with affinities similar to conventional full-size antibodies, and may possess other properties superior to conventional full-size antibodies.

The term “human antibody” herein means an antibody that comprises only human immunoglobulin protein sequences. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable region, or other combinations of sequences or subsequences. Sequence identity can be determined using the methods described herein, for example, aligning two sequences using BLAST, ALIGN, or another alignment algorithm known in the art. The corresponding human germline nucleic acid or amino acid sequence can have at least about 90%, 91%, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant” or “KD” or “M” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ m or 10⁻¹⁰ M, in some aspects, less than about 10⁻¹¹ M, 10⁻¹² M or 10⁻¹³ M.

The term “cancer” or “tumor” used herein has the broadest meaning as understood in the art and refers to the physiological condition in mammals that is typically characterized by unregulated cell growth. In the context of the present disclosure, the cancer is not limited to a certain type or location.

In the context of the present disclosure, when reference is made to an amino acid sequence, the term “conservative substitution” means substitution of the original amino acid by a new amino acid that does not substantially alter the chemical, physical, and/or functional properties of the antibody or fragment, e.g., its binding affinity to CD16A. Common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H}I interfaces or VH/VL interfaces of antibodies (see, e.g., US 2011/0287009, US2007/0178552, WO 96/027011, WO 98/050431, and Zhu et al., 1997, Protein Science, 6:781-788). In some embodiments, knob-into-holes ensure the correct pairing of two different heavy chains together during the manufacture of multi-specific antibodies. For example, multi-specific antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also ensure correct pairing.

Examples of algorithms that are suitable for determining percent sequence identity and sequence similarity are the BLAST algorithms, which are described in Altschul et al., Nuc. Acids Res. 25:3389-3402, 1977; and Altschul et al., J. Mol. Biol. 215:403-410, 1990. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89:10915), alignments (B) of 50, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, (1988), which has been incorporated into the ALIGN program (version 2.0), using a PAM120 weight residue table, a gap length penalty of 12, and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-CD16A antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g. by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination components.

The term “combination therapy” refers to the administration of two or more therapeutic agents to treat a therapeutic condition or disorder. Such administration encompasses co-administration of these therapeutic agents in a substantially simultaneous manner. Such administration also encompasses co-administration in multiple or in separate containers or formulations (e.g., capsules, powders, and liquids) for each active ingredient. Powders and/or liquids can be reconstituted or diluted to a desired dose prior to administration. In addition, “combination therapy” encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-CD16A antibody is administered to the subject at the same time as, just before, or just after administration of an additional therapeutic agent.

### DETAILED DESCRIPTION OF THE INVENTION

The present disclosure is directed to anti-CD16A antibodies and antigen-binding fragments thereof, and multi-specific antibodies or antigen-binding fragments thereof that recognize CD16A as one antigen and recognize at least one tumor antigen as at least a second antigen. The antigen binding fragments may be VH antibody fragments. The disclosed antibodies and antigen binding fragments have desirable pharmacokinetic characteristics, desirable biophysical properties, and other desirable attributes, such as selective binding of CD16A with minimal or no binding to CD16B. Pharmaceutical compositions comprising the antibodies or antigen binding fragments are also disclosed.

### Anti-CD16A Antibodies

The present disclosure provides for antibodies or antigen-binding fragments that specifically bind to CD16A. In embodiments, the anti-CD16A antibodies or antigen-binding fragments disclosed herein are able to activate human cells expressing CD16A, including NK cells, and elicit a signaling response via said binding. In embodiments, the antibodies or antigen-binding fragments are VHH antibodies. In embodiments, the antibodies or antigen-binding fragments trigger antibody-dependent cellular cytotoxicity (ADCC). In embodiments, the antibodies or antigen-binding fragments, via binding with CD16A expressing cells such as NK cells, trigger CD16A-mediated cell killing. The antibodies or antigen-binding fragments may be produced as described below.

The present disclosure provides antibodies or antigen-binding fragments that specifically bind to CD16A, wherein said antibodies or antibody fragments (e.g., antigen-binding fragments) comprise a VHH domain comprising an amino acid sequence of SEQ ID NO: 112, SEQ ID NO: 115, SEQ ID NO: 117, or SEQ ID NO: 119 (Table 2). The present disclosure also provides antibodies or antigen-binding fragments that specifically bind CD16A, wherein said antibodies or antigen-binding fragments comprise a HCDR (heavy chain complementarity determining region) comprising an amino acid sequence of any one of the HCDRs listed in Table 2. In one aspect, the present disclosure provides antibodies or antigen-binding fragments that specifically bind to CD16A, wherein said antibodies comprise (or alternatively, consist of) one, two, three, or more HCDRs comprising an amino acid sequence of any of the HCDRs listed in Table 2.

Other antibodies or antigen-binding fragments thereof of the present disclosure include amino acids that have been changed, yet have at least 60%, 70%, 80%, 90%, 95%, or 99% identity in the CDR regions with the CDR regions disclosed in Table 2. In some aspects, it includes amino acid changes wherein no more than 1, 2, 3, 4, or 5 amino acids have been changed in the CDR regions when compared with the CDR regions depicted in the sequences in Table 2.

Other antibodies of the present disclosure include those where the amino acids or nucleic acids encoding the amino acids have been changed, yet have at least 60%, 70%, 80%, 90%, 95%, or 99% identity to the sequences disclosed in Table 2. In some aspects, it includes changes in the amino acid sequences wherein no more than 1, 2, 3, 4, or 5 amino acids have been changed in the variable regions when compared with the variable regions depicted in the sequences disclosed in Table 2, while retaining substantially the same therapeutic activity.

The present disclosure also provides nucleic acid sequences that encode VH domain antibodies that specifically bind to CD16A and the full-length heavy chain of the antibodies. Such nucleic acid sequences can be optimized for expression in mammalian cells.

Identification of Epitopes and Antibodies that Bind to the Same Epitope

The present disclosure provides antibodies and antigen-binding fragments that bind to an epitope of human CD16A. In certain aspects, the antibodies and antigen-binding fragments can bind to the same epitope of CD16A.

The present disclosure also provides for antibodies and antigen-binding fragments thereof that bind to the same epitope as do the anti-CD16A antibodies described in Table 2. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CD16A demonstrates that the test antibody can compete with that antibody or antigen-binding fragment thereof for binding to CD16A. Such an antibody can, without being bound to any one theory, bind to the same or a related (e.g., a structurally similar or spatially proximal) epitope on CD16A as the antibody or antigen-binding fragment thereof with which it competes. In a certain aspect, the antibody that binds to the same epitope on CD16A as the antibodies or antigen-binding fragments thereof of the present disclosure is a human or humanized monoclonal antibody. Such human or humanized monoclonal antibodies can be prepared and isolated as described herein.

### Alteration of the Fc Region

In aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII, and FcRn have been mapped and variants with improved binding have been described (see Shields et al., J. Biol. Chem. 276:6591-6604, 2001).

In still another aspect, the glycosylation of the multi-specific antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for an antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277.26733-26740). WO99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures, which results in increased ADCC activity of the antibodies (see also Umana et al., Nat. Biotech. 17:176-180, 1999).

### Anti-CD16A Antibody Production

Anti-CD16A antibodies, antigen-binding fragments and multi-specific antibodies can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 113, SEQ ID NO: 116, SEQ ID NO: 118, and SEQ ID NO: 120.

The polynucleotides of the present disclosure can encode the variable region sequence of an anti-CD16A antibody. They can also encode both a variable region and a constant region of the antibody. Some of the polynucleotide sequences encode a polypeptide that comprises variable regions of both the heavy chain and the light chain of one of the exemplified anti-CD16A antibodies.

Also provided in the present disclosure are expression vectors and host cells for producing the anti-CD16A antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-CD16A antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-CD16A antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-CD16A antibody vectors can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-CD16A polypeptides. Insect cells in combination with baculovirus vectors can also be used.

In other aspects, mammalian host cells are used to express and produce the anti-CD16A polypeptides of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Anti-CD16A Multi-Specific Antibodies and Constructs

In one embodiment, the anti-CD16A antibodies or antigen binding fragments disclosed herein can be incorporated into an anti-CD16AxTAA multi-specific antibody, wherein TAA is an antibody or fragment thereof directed to any human tumor associated antigen (TAA). An antibody is a multi-specific antibody molecule if, for example, it comprises a number of antigen binding domains, wherein at least one antigen binding domain sequence specifically binds CD16A and a second antigen binding domain sequence specifically binds a TAA. In embodiments, the multi-specific antibody comprises a third, fourth, or fifth antigen binding domain. In embodiments, the multi-specific antibody is a bispecific antibody, a tri-specific antibody, or tetra-specific antibody. In each embodiment, the multi-specific antibody comprises at least one anti-CD16A antigen binding domain and at least one anti-TAA antigen binding domain. In embodiments, the binding to CD16A of the multi-specific antibodies or fragments thereof via the anti-CD16A binding domain arm may lead to the activation of NK cells and killing of tumor cells that express the antigen for another arm(s) of said multi-specific antibodies or fragments thereof via the binding therewith. In this way, the anti-CD16A multi-specific antibody may be used in the treatment of various cancers or other diseases, depending on the specificity of the multi-specific antibody's other arm(s).

In one embodiment, the multi-specific antibody is a bispecific antibody. As used herein, a bispecific antibody specifically binds only two antigens. The bispecific antibody comprises a first antigen binding domain that specifically binds CD16A and a second antigen binding domain that specifically binds a TAA. Included is a bispecific antibody comprising a heavy chain variable domain that specifically binds CD16A and a heavy chain variable domain and a light chain variable domain that specifically bind a TAA. In another embodiment, the bispecific antibody comprises an antigen binding fragment of an antibody that specifically binds CD16A and an antigen binding fragment that specifically binds a TAA. When the bispecific antibody comprises antigen binding fragments, the antigen-binding fragment can be a Fab, F(ab′)2, Fv, or a single chain Fv (scFv).

Previous experimentation (Coloma and Morrison, Nature Biotech. 15: 159-163 (1997)) described a tetravalent bispecific antibody that was engineered by fusing DNA encoding a single chain anti-dansyl antibody Fv (scFv) after the C terminus (CH3-scFv) or after the hinge (hinge-scFv) of an IgG3 anti-dansyl antibody. The present disclosure provides multivalent antibodies (e.g. tetravalent antibodies) with at least two antigen binding domains, that can be readily produced by recombinant expression of nucleic acid encoding the polypeptide chains of the antibody. The multivalent antibody herein comprises three to eight, but preferably four, antigen binding domains that specifically bind at least two antigens.

The disclosure provides for a bispecific tetravalent antibody comprising VD1-CL-(X1)n-VD2-CH1-Fc or VD1-CH-(X1)n-VD2-CL-Fc, wherein VD1 is a first variable domain, VD2 is a second variable domain, Fc is one polypeptide chain of an Fc region, CH or CL is a constant heavy or constant light domain, respectively, and (X1)n is a linker of at least 2 amino acids.

In one embodiment, the bispecific tetravalent antibody can be multimer of four polypeptide chains, two heavy chains each comprising a first VH domain (VH1), a first CH1 domain, a second VH domain (VH2), an Fc region comprising a second CH1, hinge, CH2, a CH3 and two light chains, each light chain comprising a first VL domain (VL1), a first CL region, a second VL domain (VL2), and a second CL region. In another embodiment, the bispecific tetravalent antibody can comprise multiple antibody Fab fragments linked together to a single Fc domain. For example, a Fab1 can be linked via a polypeptide linker to a Fab2, which comprises the CH1 domain of one of the Fab, hinge region, then CH2 and CH3 of the Fc domain. For example, an anti-TAA Fab can be linked via a linker from the CL domain of the anti-TAA Fab to a VH domain of anti-CD16A Fab and from the CH1 domain of the anti-CD16A Fab, the hinge region, CH2 and CH3 domains. In another example, an anti-CD16A Fab can be linked via a linker from the CL domain of the anti-CD16A Fab to a VHH domain of anti-TAA Fab and from the CH1 domain of the anti-TAA Fab, the hinge region, CH2 and CH3 domains.

In one embodiment, the anti-CD16A antibodies or antigen-binding fragments disclosed herein can be incorporated into a construct. The antibody or antigen-binding fragment in a construct may be a VHH. In some examples, the VHH is combined with a second antigen-binding fragment. In some examples, the VHH is combined with a payload, such as a therapeutic, or a tool, such as a visualization agent. The construct may be a multi-specific antibody, a heavy chain antibody, a bivalent VHH, a biparatopic VHH, a bispecific VHH, a VHH-scFv, a VHH-cytokine, a VHH-drug, a VHH-nanoparticles, a VHH-virus, or a VHH-imaging probe; construct is a multi-specific antibody; multi-specific antibody is a bispecific antibody.

### Linkers

The domains and/or regions of the polypeptide chains of the bispecific tetravalent antibodies or constructs disclosed herein may be separated by linker regions of various lengths. In some embodiments, the antigen binding domains are separated from each other, a CL, CH1, hinge, CH2, CH3, or the entire Fc region by a linker region. For example, the polypeptide chains may include the sequence VL1-CL-(linker) VH2-CH1, VH-linker-VL. Such linker regions may comprise a random assortment of amino acids or a restricted set of amino acids. Such linker regions can be flexible or rigid (see US2009/0155275).

Multi-specific antibodies have been constructed by genetically fusing two single chain Fv (scFv) or Fab fragments with or without the use of flexible linkers (Mallender et al., J. Biol. Chem. 1994 269:199-206; Mack et al., Proc. Natl. Acad. Sci. USA. 1995 92:7021-5; Zapata et al., Protein Eng. 1995 8.1057-62); via a dimerization device such as leucine zipper (Kostelny et al., J. Immunol. 1992148:1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8; Zhu et al., Bio/Technology (NY) 1996 14:192-6); Fab-scFv fusion (Schoonjans et al., J. Immunol. 2000 165:7050-7); and mini antibody formats (Pack et al., Biochemistry 1992.31:1579-84; Pack et al., Bio/Technology 1993 11:1271-7).

The multi-specific antibodies and constructs as disclosed herein may include a linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of their antigen binding domains, CL domains, CH1 domains, hinge regions, CH2 domains, CH3 domains, or Fc regions. In some embodiments, the linker region is comprised of the amino acids glycine and serine. The linker may include the sequence GS (SEQ ID NO: 239), GGS (SEQ ID NO: 240), GSG (SEQ ID NO: 241), SGG (SEQ ID NO: 242), GGG (SEQ ID NO: 243), GGGS (SEQ ID NO: 244), SGGG (SEQ ID NO: 245), GGGGS (SEQ ID NO: 246), CGGGSGS (SEQ ID NO: 247), GGGGSGS (SEQ ID NO: 248), GCGGGSGGS (SEQ ID NO: 249), GGGGSGGGGS (SEQ ID NO: 250), GGGGSGGGGSGGGGS (SEQ ID NO: 251), AKTTPKLEEGEFSEAR (SEQ ID NO: 252), AKTTPKLEEGEFSEARV (SEQ ID NO: 253), AKTTPKLGG (SEQ ID NO: 254), SAKTTPKLGG (SEQ ID NO: 255), AKTTPKLEEGEFSEARV (SEQ ID NO: 256), SAKTTP (SEQ ID NO: 257), SAKTTPKLGG (SEQ ID NO: 258), RADAAP (SEQ ID NO: 259), RADAAPTVS (SEQ ID NO: 260), RADAAAAGGPGS (SEQ ID NO: 261), RADAAAA(G₄S)₄ (SEQ ID NO: 262), SAKTTP (SEQ ID NO: 263), SAKTTPKLGG (SEQ ID NO: 264), SAKTTPKLEEGEFSEARV (SEQ ID NO: 265), ADAAP (SEQ ID NO: 266), ADAAPTVSIFPP (SEQ ID NO: 267), TVAAP (SEQ ID NO: 268), TVAAPSVFIFPP (SEQ ID NO: 269), QPKAAP (SEQ ID NO: 270), QPKAAPSVTLFPP (SEQ ID NO: 271), AKTTPP (SEQ ID NO: 272), AKTTPPSVTPLAP (SEQ ID NO: 273), AKTTAP (SEQ ID NO: 274), AKTTAPSVYPLAP (SEQ ID NO: 275), ASTKGP (SEQ ID NO: 276), ASTKGPSVFPLAP (SEQ ID NO: 277), GENKVEYAPALMALS (SEQ ID NO: 278), GPAKELTPLKEAKVS (SEQ ID NO: 279), and GHEAAAVMQVQYPAS (SEQ ID NO: 280) or any combination thereof (see WO2007/024715).

### Dimerization-Specific Amino Acids

In one embodiment, the multivalent antibodies or constructs comprises at least one dimerization-specific amino acid change. The dimerization-specific amino acid change may result in “knob-into-hole” interactions, and may increase the likelihood of correct assembly of desired multivalent antibodies. The dimerization-specific amino acids may be within the CH1 domain or the CL domain or combinations thereof. Suitable dimerization-specific amino acids used to pair CH1 domains with other CH1 domains (CH1-CH1) and CL domains with other CL domains (CL-CL) may be found at least in the disclosures of WO2014082179, WO2015181805, and WO2017059551. The dimerization-specific amino acids can also be within the Fc domain and can be in combination with dimerization-specific amino acids within the CH1 or CL domains. In one embodiment, the present disclosure provides a bispecific antibody comprising at least one dimerization-specific amino acid pair.

### Methods of Detection and Diagnosis

The antibodies or antigen-binding fragments of the present disclosure are useful in a variety of applications including, but not limited to, methods for the detection of CD16A. In one aspect, the antibodies or antigen-binding fragments are useful for detecting the presence of CD16A in a biological sample. The term “detecting” as used herein includes quantitative or qualitative detection. In certain aspects, a biological sample comprises a cell or tissue. In other aspects, such tissues include normal and/or cancerous tissues that express CD16A at higher levels relative to other tissues.

In one aspect, the present disclosure provides a method of detecting the presence of CD16A in a biological sample. In certain aspects, the method comprises contacting the biological sample with an anti-CD16A antibody or antigen binding fragment thereof under conditions permissive for binding of the antibody to an antigen and detecting whether a complex is formed between the antibody and the antigen. The biological sample can include, without limitation, urine, tissue, sputum, or blood.

Also included is a method of diagnosing a disorder associated with expression of CD16A. In certain aspects, the method comprises contacting a test cell with an anti-CD16A antibody or antigen binding fragment; determining the level of expression (either quantitatively or qualitatively) of CD16A expressed by the test cell by detecting binding of the anti-CD16A antibody or antigen binding fragment to the CD16A polypeptide; and comparing the level of expression by the test cell with the level of CD16A expression in a control cell (e.g., a normal cell of the same tissue origin as the test cell or a non-CD16A expressing cell), wherein a higher level of CD16A expression in the test cell as compared to the control cell indicates the presence of a disorder associated with expression of CD16A.

### Pharmaceutical Compositions and Formulations

Also provided are compositions, including pharmaceutical formulations, comprising an anti-CD16A antibody, antigen binding fragment thereof, multi-specific antibody, or polynucleotides comprising sequences encoding an anti-CD16A antibody, antigen binding fragment thereof, or multi-specific antibody. These compositions can further comprise suitable carriers, such as pharmaceutically acceptable excipients including buffers, which are well known in the art.

Pharmaceutical formulations of an anti-CD16A antibody or antigen binding fragment thereof as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility can be readily accomplished, e.g., by filtration through sterile filtration membranes.

### Equivalent

It is to be understood that, while the anti-human 4Ig-B7H3 antibodies and antigen binding fragments thereof have been described in conjunction with a detailed description thereof, the foregoing description is intended to illustrate and not limit the scope of the invention, which is defined by the scope of the appended claims. Other aspects, advantages, and modifications are within the scope of the claims below.

It is to be understood that one, some, any, or all of the features of the various embodiments disclosed herein may be combined to form further embodiments of the present disclosure. These and other aspects of the present disclosure will be apparent to those skilled in the art.

### Sequence List

The sequences list of the present disclosure is provided below in Table 1 and 2.

TABLE 1
CD16A and CD16B Sequences

| Name | SEQ ID | Sequence |
|---|---|---|
| Human | SEQ ID NO: | MWQLLLPTALLLLVSAGMRTEDLPKAVVFLEPQWYRVL |
| CD16A | 101 | EKDSVTLKCQGAYSPEDNSTQWFHNESLISSQASSYFIDA |
| (V158) | | ATVDDSGEYRCQTNLSTLSDPVQLEVHIGWLLLQAPRW |
| | | VFKEEDPIHLRCHSWKNTALHKVTYLQNGKGRKYFHHN |
| | | SDFYIPKATLKDSGSYFCRGLVGSKNVSSETVNITITQGL |
| | | AVSTISSFFPPGYQVSFCLVMVLLFAVDTGLYFSVKTNIRS |
| | | STRDWKDHKFKWRKDPQDK |
| Human | SEQ ID NO: | MWQLLLPTALLLLVSAGMRTEDLPKAVVFLEPQWYRVL |
| CD16A | 102 | EKDSVTLKCQGAYSPEDNSTQWFHNESLISSQASSYFIDA |
| (F158) | | ATVDDSGEYRCQTNLSTLSDPVQLEVHIGWLLLQAPRW |
| | | VFKEEDPIHLRCHSWKNTALHKVTYLQNGKGRKYFHHN |
| | | SDFYIPKATLKDSGSYFCRGLFGSKNVSSETVNITITQGLA |
| | | VSTISSFFPPGYQVSFCLVMVLLFAVDTGLYFSVKTNIRSS |
| | | TRDWKDHKFKWRKDPQDK |
| Human | SEQ ID NO: | MWQLLLPTALLLLVSAGMRTEDLPKAVVFLEPQWYRVL |
| CD16B | 103 | EKDSVTLKCQGAYSPEDNSTQWFHNENLISSQASSYFID |
| (NA1) | | AATVDDSGEYRCQTNLSTLSDPVQLEVHVGWLLLQAPR |
| | | WVFKEEDPIHLRCHSWKNTALHKVTYLQNGKDRKYFH |
| | | HNSDFHIPKATLKDSGSYFCRGLVGSKNVSSETVNITITQ |
| | | GLAVSTISSFSPPGYQVSFCLVMVLLFAVDTGLYFSVKTNI |
| Human | SEQ ID NO: | MWQLLLPTALLLLVSAGMRTEDLPKAVVFLEPQWYSVL |
| CD16B | 104 | EKDSVTLKCQGAYSPEDNSTQWFHNESLISSQASSYFIDA |
| (NA2) | | ATVNDSGEYRCQTNLSTLSDPVQLEVHIGWLLLQAPRW |
| | | VFKEEDPIHLRCHSWKNTALHKVTYLQNGKDRKYFHHN |
| | | SDFHIPKATLKDSGSYFCRGLVGSKNVSSETVNITITQGL |
| | | AVSTISSFSPPGYQVSFCLVMVLLFAVDTGLYFSVKTNI |
| Human | SEQ ID NO: | MWQLLLPTALLLLVSAGMRTEDLPKAVVFLEPQWYSVL |
| CD16B | 105 | EKDSVTLKCQGAYSPEDNSTQWFHNENLISSQASSYFID |
| (SH) | | AATVNDSGEYRCQTNLSTLSDPVQLEVHIGWLLLQAPR |
| | | WVFKEEDPIHLRCHSWKNTALHKVTYLQNGKDRKYFH |
| | | HNSDFHIPKATLKDSGSYFCRGLVGSKNVSSETVNITITQ |
| | | GLAVSTISSFSPPGYQVSFCLVMVLLFAVDTGLYFSVKTNI |
| Cyno | SEQ ID NO: | MWQLLLPTALLLLVSAGMRAEDLPKAVVFLEPQWYRVL |
| CD16 | 106 | EKDRVTLKCQGAYSPEDNSTRWFHNESLISSQTSSYFIAA |
| | | ARVNNSGEYRCQTSLSTLSDPVQLEVHIGWLLLQAPRW |
| | | VFKEEESIHLRCHSWKNTLLHKVTYLQNGKGRKYFHQN |
| | | SDFYIPKATLKDSGSYFCRGLIGSKNVSSETVNITITQDLA |
| | | VSSISSFFPPGYQVSFCLVMVLLFAVDTGLYFSMKKSIPSS |
| | | TRDWEDHKFKWSKDPQDK |
| human | SEQ ID NO: | EPKSSDKTHTCPPCPAPPAAGPSVFLFPPKPKDTLMISRTP |
| IgG1 | 107 | EVTCVVVDVSHEDPEVKFNWYVDGVEVHNAKTKPREE |
| mf Fc | | QYNSTYRVVSVLTVLHQDWLNGKEYKCKVSNKALAAPI |
| | | EKTISKAKGQPREPQVYTLPPSREEMTKNQVSLTCLVKG |
| | | FYPSDIAVEWESNGQPENNYKTTPPVLDSDGSFFLYSKLT |
| | | VDKSRWQQGNVFSCSVMHEALHNHYTQKSLSLSPGK |
| LS21 | SEQ ID NO: | EVOLVQSGAEVKKPGESLKVSCKASGYTFTSYYMHWVR |
| | 108 | QAPGQGLEWMGIINPSGGSTSYAQKFQGRVTMTRDTSTS |
| | | TVYMELSSLRSEDTAVYYCARGSAYYYDFADYWGQGTL |
| | | VTVSSGGGGSGGGGSGGGGSQPVLTQPSSVSVAPGQTA |
| | | TISCGGHNIGSKNVHWYQQRPGQSPVLVIYQDNKRPSGI |
| | | PERFSGSNSGNTATLTISGTQAMDEADYYCQVWDNYSV |
| | | LFGGGTKLTVL |

TABLE 2
Anti-CD16A VHH Sequences

| Antibody | SEQ ID NO | Note | Sequence |
|---|---|---|---|
| BG523P | SEQ ID NO: 109 | CDR1 | SYAVG |
| | | (Kabat) | |
| | SEQ ID NO: 110 | CDR2 | AIRRSDGTTDYVDSVKG |
| | | (Kabat) | |
| | SEQ ID NO: 111 | CDR3 | SDSDYEPLGDY |
| | | (Kabat) | |
| | SEQ ID NO: 112 | VHH AA | QVQLVESGGGVVQPGGSLTLSCEASGRT |
| | | | FGSYAVGWFRQALGRGREFVAAIRRSDG |
| | | | TTDYVDSVKGRFTISRDNAKNTVYLQM |
| | | | NSLKPEDTAVYYCAASDSDYEPLGDYW |
| | | | GQGTQVTVSS |
| | SEQ ID NO: 113 | VHH | CAGGTGCAGCTCGTGGAGTCTGGGGG |
| | | DNA | CGGCGTGGTGCAGCCTGGGGGGTCTCT |
| | | | GACACTCTCCTGTGAAGCCTCTGGACG |
| | | | CACCTTCGGCAGCTATGCCGTGGGCTG |
| | | | GTTCCGCCAGGCTCTTGGGAGGGGGCG |
| | | | TGAGTTTGTAGCAGCTATTAGGCGGAG |
| | | | TGATGGTACTACAGACTATGTAGACTCC |
| | | | GTGAAGGGCCGATTCACCATCTCCAGA |
| | | | GACAACGCCAAGAACACGGTGTATCTG |
| | | | CAAATGAACAGCCTGAAACCTGAGGA |
| | | | CACGGCCGTTTATTACTGTGCAGCATCG |
| | | | GATAGCGACTATGAACCCTTAGGGGAC |
| | | | TACTGGGGCCAGGGGACCCAGGTCAC |
| | | | CGTCTCCTCA |
| BG524P | SEQ ID NO: 109 | CDR1 | SYAVG |
| | | (Kabat) | |
| | SEQ ID NO: 114 | CDR2 | AIRRSDATTDYVDSVKG |
| | | (Kabat) | |
| | SEQ ID NO: 111 | CDR3 | SDSDYEPLGDY |
| | | (Kabat) | |
| | SEQ ID NO: 115 | VHH AA | EVQLVESGGGLVQPGGSLRLSCAASGRT |
| | | | FGSYAVGWFRQAPGKGREWVAAIRRSD |
| | | | ATTDYVDSVKGRFTISRDNAKNSVYLQ |
| | | | MNSLRPEDTAVYYCAASDSDYEPLGDY |
| | | | WGQGTLVTVSS |
| | SEQ ID NO: 116 | VHH | GAGGTGCAGCTCGTGGAATCCGGGGG |
| | | DNA | CGGGCTGGTGCAACCCGGCGGGAGCC |
| | | | TGAGACTGAGCTGCGCCGCTAGCGGCA |
| | | | GAACCTTCGGCAGCTACGCCGTGGGCT |
| | | | GGTTCAGACAAGCCCCCGGCAAGGGC |
| | | | AGAGAGTGGGTGGCCGCCATCAGAAG |
| | | | AAGCGACGCCACCACCGACTACGTGG |
| | | | ACAGCGTGAAGGGCAGATTCACCATCA |
| | | | GCAGAGACAACGCCAAGAACAGCGTG |
| | | | TACCTGCAGATGAACAGCCTGAGACCC |
| | | | GAGGACACCGCCGTGTACTACTGCGCC |
| | | | GCCTCCGACAGCGACTACGAGCCTCTG |
| | | | GGCGATTACTGGGGCCAAGGCACCCTG |
| | | | GTGACCGTGAGCAGC |
| BG525P | SEQ ID NO: 109 | CDR1 | SYAVG |
| | | (Kabat) | |
| | SEQ ID NO: 110 | CDR2 | AIRRSDGTTDYVDSVKG |
| | | (Kabat) | |
| | SEQ ID NO: 111 | CDR3 | SDSDYEPLGDY |
| | | (Kabat) | |
| | SEQ ID NO: 117 | VHH AA | EVQLVESGGGLVQPGGSLRLSCAASGRT |
| | | | FGSYAVGWFRQAPGRGREFVAAIRRSDG |
| | | | TTDYVDSVKGRFTISRDNAKNTVYLQM |
| | | | NSLKPEDTAVYYCAASDSDYEPLGDYW |
| | | | GQGTLVTVSS |
| | SEQ ID NO: 118 | VHH | GAGGTGCAGCTCGTGGAATCCGGGGG |
| | | DNA | CGGGCTGGTGCAACCCGGCGGGAGCC |
| | | | TGAGACTGAGCTGCGCCGCTAGCGGCA |
| | | | GAACCTTCGGCAGCTACGCCGTGGGCT |
| | | | GGTTCAGACAAGCCCCCGGCAgaGGCA |
| | | | GAGAGTtcGTGGCCGCCATCAGAAGAA |
| | | | GCGACGgCACCACCGACTACGTGGACA |
| | | | GCGTGAAGGGCAGATTCACCATCAGCA |
| | | | GAGACAACGCCAAGAACACCGTGTACC |
| | | | TGCAGATGAACAGCCTGAagCCCGAGG |
| | | | ACACCGCCGTGTACTACTGCGCCGCCT |
| | | | CCGACAGCGACTACGAGCCTCTGGGCG |
| | | | ATTACTGGGGCCAAGGCACCCTGGTGA |
| | | | CCGTGAGCAGC |
| BG526P | SEQ ID NO: 109 | CDR1 | SYAVG |
| | | (Kabat) | |
| | SEQ ID NO: 114 | CDR2 | AIRRSDATTDYVDSVKG |
| | | (Kabat) | |
| | SEQ ID NO: 111 | CDR3 | SDSDYEPLGDY |
| | | (Kabat) | |
| | SEQ ID NO: 119 | VHH AA | EVQLVESGGGLVQPGGSLRLSCAASGRT |
| | | | FGSYAVGWFRQAPGKGREFVAAIRRSDA |
| | | | TTDYVDSVKGRFTISRDNAKNTVYLQM |
| | | | NSLKPEDTAVYYCAASDSDYEPLGDYW |
| | | | GQGTLVTVSS |
| | SEQ ID NO: 120 | VHH | GAGGTGCAGCTCGTGGAATCCGGGGG |
| | | DNA | CGGGCTGGTGCAACCCGGCGGGAGCC |
| | | | TGAGACTGAGCTGCGCCGCTAGCGGCA |
| | | | GAACCTTCGGCAGCTACGCCGTGGGCT |
| | | | GGTTCAGACAAGCCCCCGGCAAGGGC |
| | | | AGAGAGTTCGTGGCCGCCATCAGAAG |
| | | | AAGCGACGCCACCACCGACTACGTGG |
| | | | ACAGCGTGAAGGGCAGATTCACCATCA |
| | | | GCAGAGACAACGCCAAGAACACCGTG |
| | | | TACCTGCAGATGAACAGCCTGAAGCCC |
| | | | GAGGACACCGCCGTGTACTACTGCGCC |
| | | | GCCTCCGACAGCGACTACGAGCCTCTG |
| | | | GGCGATTACTGGGGCCAAGGCACCCTG |
| | | | GTGACCGTGAGCAGC |
| CB6 | SEQ ID NO: 121 | Heavy | EVQLVESGGGLVQPGGSLRLSCAASGFT |
| | | chain | VSSNYMSWVRQAPGKGLEWVSVIYSGG |
| | | | STFYADSVKGRFTISRDNSMNTLFLQMN |
| | | | SLRAEDTAVYYCARVLPMYGDYLDYWG |
| | | | QGTLVTVSSASTKGPSVFPLAPSSKSTSG |
| | | | GTAALGCLVKDYFPEPVTVSWNSGALTS |
| | | | GVHTFPAVLQSSGLYSLSSVVTVPSSSLG |
| | | | TQTYICNVNHKPSNTKVDKRVEPKSCDK |
| | | | THTCPPCPAPELLGGPSVFLFPPKPKDTL |
| | | | MISRTPEVTCVVVDVSHEDPEVKFNWY |
| | | | VDGVEVHNAKTKPREEQYNSTYRVVSV |
| | | | LTVLHQDWLNGKEYKCKVSNKALPAPIE |
| | | | KTISKAKGQPREPQVYTLPPSRDELTKNQ |
| | | | VSLTCLVKGFYPSDIAVEWESNGQPENN |
| | | | YKTTPPVLDSDGSFFLYSKLTVDKSRWQ |
| | | | QGNVFSCSVMHEALHNHYTQKSLSLSP |
| | | | GK |
| | SEQ ID NO: 122 | Light | DIVMTQSPSSLSASVGDRVTITCRASQSIS |
| | | chain | RYLNWYQQKPGKAPKLLIYAASSLQSGV |
| | | | PSRFSGSGSGTDFTLTISSLQPEDFATYYC |
| | | | QQSYSTPPEYTFGQGTKLEIKRTVAAPSV |
| | | | FIFPPSDEQLKSGTASVVCLLNNFYPREA |
| | | | KVQWKVDNALQSGNSQESVTEQDSKDS |
| | | | TYSLSSTLTLSKADYEKHKVYACEVTHQ |
| | | | GLSSPVTKSFNRGEC |

### EXAMPLES

### Example 1. Generation of Anti-CD16A VHH

### Human CD164 Recombinant Protein and Cell Lines for Immunization and Assay

A recombinant six-histidine-tagged extracellular domain (ECD) fragment of human CD16A protein (V158) (SEQ ID NO: 101)—referred to as human CD16A-His₆ (V158)—was purchased from a commercial source (Sino Biologics) and utilized as the antigen to immunize an alpaca. Recombinant six-histidine-tagged ECD fragments of human CD16A (F7158) (SEQ ID NO: 102), human CD16B (NA1) (SEQ ID NO: 103), human CD16B (NA2) (SEQ ID NO: 104), human CD16B (SH) (SEQ ID NO: 105), cyno CD16 (SEQ ID NO: 106)—referred to as human CD16A-His₆ (F158), human CD16B-His₆ (NA1), human CD16B-His₆ (NA2), human CD16B-His₆ (SH), and cyno CD16-His₆—respectively were purchased from a commercial source (Sino Biologics) and used for various in vitro assays.

To facilitate screening and detection, a DNA fragment for human CD16A (V158) ECD (AA 1-208 of SEQ ID NO: 101) was fused with a C-terminal human IgG1 mf Fc tag (SEQ ID NO: 107), mouse IgG2a Fc tag, or alpaca IgG2b Fc tag and subjected to transient expression in Expi293 cells (Thermofisher Scientific). Culture supernatant was harvested and clarified, and affinity purified with a Protein A column (Cytiva). The final products were buffer exchanged to DPBS by ultrafiltration/diafiltration (UF/DF), and stored at −80° C.

To evaluate the binding activity of the antibody to CD16A expressed on living cells, NK92mi (ATCC, CRL-2407) cells were engineered to over-express human CD16A (NK92mi/CD16A F158 and NK92mi/CD16A V158) by co-transducing expression plasmids containing CD16A (F158 or V158) and FcRγ cDNAs. NK92mi/CD16B (NA1) and NK92mi/CD16B (NA2) expressing cell lines were prepared similarly from CD16B (NA1) or CD16B (NA2) expression plasmids.

### Immunization and Screening

One alpaca was immunized with recombinant protein human CD16A-His₆ (V158) as the antigen by an external contract research organization and immune VHH phage library was constructed from isolated alpaca peripheral blood mononuclear cells (PBMC) (Pardon Els et al. (2014) Nature Protocols) after the third immunization. Phage display selection was carried out using standard protocols (Silacci et al., (2005) Proteomics, 5, 2340-50; Zhao et al., (2014) PLoS One, 9, e111339). In brief, 10 mg/ml of immobilized human CD16A-V158-AlpacaIgG2b in immunotube was utilized to enrich human CD16A(V158) specific binders in panning round 1 and 2. Immunotube was blocked with 5% milk powder (w/v) in PBS supplemented with 1% Tween 20 (MPBST) for 1 h. After washing with PBST (PBS buffer supplemented with 0.05% Tween 20), 1×10¹³ (round 1) or 2×10¹² (round 2) phages were initially depleted by human CD16B-His₆ (NA2) in MPBST for 1 hour and then incubated with the antigen for 1 hour. After washing with PBST, bound phages were eluted with 100 mM triethylamine (Sigma-Aldrich). Eluted phages were used to infect mid-log phase E. coli TG1 bacteria and plated onto 2×YT (Yeast Extract Tryptone)-agar plates supplemented with 2% glucose and 100 μg/mL ampicillin. After three rounds of selections, individual clones were picked up and phage-containing supernatants were prepared using standard protocols. Phage ELISA was used to screen for anti-human CD16A antibodies.

For phage ELISA, a Maxisorp immunoplate was coated with recombinant protein human CD16A-His₆ (V158) as antigen and blocked with 5% milk powder (w/v) in PBS buffer. Phage supernatant was blocked with MPBST for 30 min and added to wells of the ELISA plate for 1 hour. After washing with PBST, bound phage was detected using HRP-conjugated anti-M13 antibody (GE Healthcare) and 3,3′,5,5′-tetramethylbenzidine substrate (Cat.: 00-4201-56, eBioscience, USA).

Positive clones from phage ELISA were sequenced and recovered. Six anti-CD16A VHH variants were constructed by fusing their open reading frames with a C-terminal human IgG1 mf Fc (SEQ ID NO: 107) tag eukaryotic expression vector. The plasmids were transfected into ExpiCHO-s cells (Thermofisher Scientific) using MAX Titer protocol. The Fc-tagged VHH variants (VHH-Fc) were purified by MabSelect SuRe (Cytiva), followed by SPHP column (Cytiva). The final products were buffer exchanged to DPBS by UF/DF and stored at −80° C. for later use, including binding analysis.

For antigen ELISA, a Maxisorp immunoplate was coated with antigens (human CD16A (V158), human CD16A (F158), human CD16B (NA1), human CD16B (NA2), human CD16B (SH), or cyno CD16) and blocked with 3% BSA (w/v) in PBS buffer (blocking buffer). Monoclonal VHH-Fc antibodies were blocked with blocking buffer for 30 min and added to wells of the ELISA plate for 1 h. After washes with PBST, bound antibodies were detected using HRP-conjugated anti-human IgG antibody (Sigma, A0170) and 3,3′,5,5′-tetramethylbenzidine substrate (Cat.: 00-4201-56, eBioscience, USA).

For flow cytometry, NK92mi/CD16A V158 cells, NK92mi/CD16B(NA1) cells and NK92mi/CD16B(NA2) cells (10⁵ cells/well) were incubated with various concentrations of IgG-like antibodies, followed by binding with Alexa Fluro-647-labeled anti-human IgG Fc antibody (Cat.: 409320, BioLegend, USA). Cell fluorescence was quantified using a flow cytometer (Guava easyCyte™ 8HT, Merck-Millipore, USA).

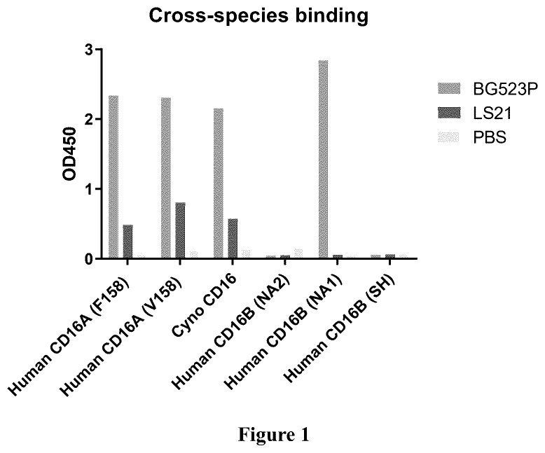

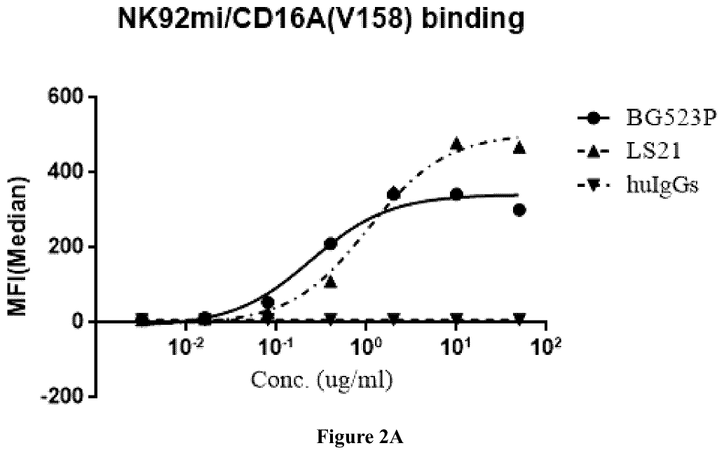

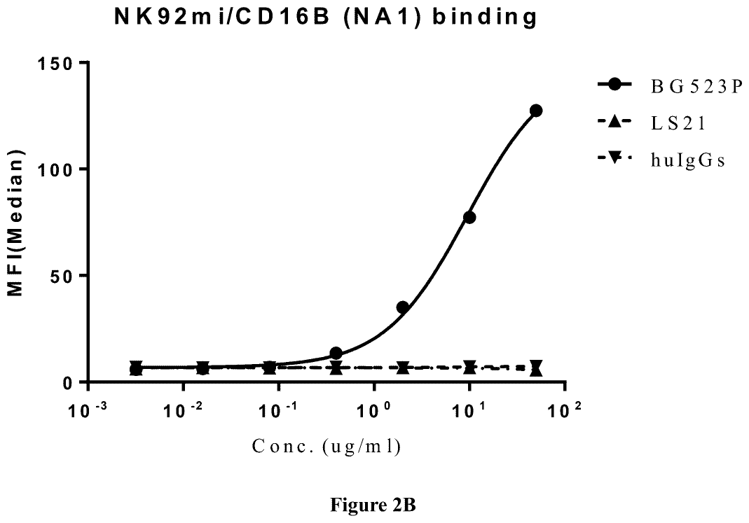

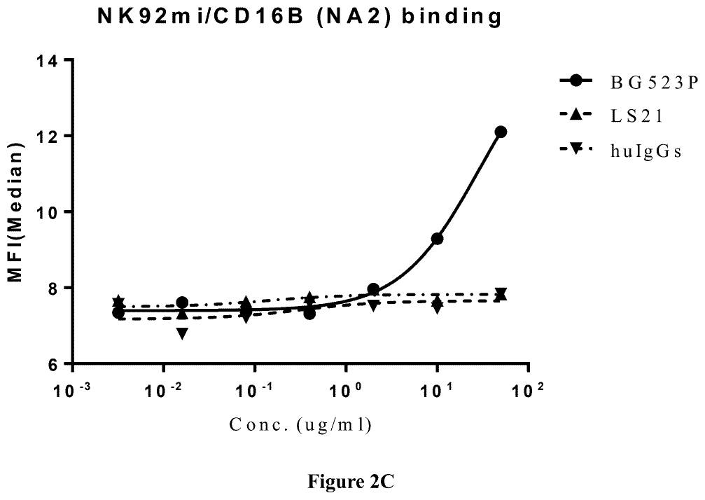

Following the procedures disclosed above, 44 positive clones were sequenced and recovered, and one representative positive anti-CD16A variant BG523P (VHH AA SEQ ID NO: 112, VHH DNA SEQ ID NO: 113) was obtained from six VHH-Fc fusion clones. The binding affinities to CD16A of BG523P was also confirmed by antigen ELISA. The ELISA and FACS analysis results of BG523P vs a positive control, LS21, are shown in Tables 3, 4, and 5, and FIGS. 1 and 2A-2C. More specifically, data for FACS binding to NK92mi/CD16A cell lines of BG523P vs. a human CD16A specific binder LS21 as a positive control (SEQ ID NO: 108, Patent. EP1888645B1) are given in Table 3, which shows BG523P has specificity for binding with NK92mi/CD16A cells. FIG. 1 illustrates that BG523P, compared to LS21, showed a higher binding with human CD16A, CD16B (NA1), and cyno CD16 at 1 μg/ml. FIG. 2A shows BG523P specifically binds with NK92mi/CD16A cells. FIGS. 2A and 2B show BG523P exhibits weak binding with NK92mi/CD16B at high concentrations. Although BG523P exhibits a slight binding activity to CD16B (NA1) in an ELISA assay as well as in a FACS assay (FIG. 1, Table 4), its binding affinity was significantly reduced (the calculated EC₅₀ value was decreased about 40-fold in comparison to that of CD16A binding in a FACS assay). This result suggests that BG523P could selectively bind to CD16A over CD16B. FIG. 1 also shows that BG523P barely binds to CD16B SH allotype. Considering that the predominant variants for human CD16B are NA1 and NA2 allotypes and the frequency for SH allotype is rare and reported to be less than 0.05 in Caucasians, binding properties toward CD16B SH allotype were not further characterized.

TABLE 3
FACS based binding of VHH to NK92mi/CD16A cell lines

| | BG523P | LS21 | Human IgG |
|---|---|---|---|
| EMAX | 342.0 | 504.8 | 7.55 |
| EC₅₀ (μg/mL) | 0.242 | 1.041 | 0.01122 |

TABLE 4
FACS based binding of VHH to NK92mi/CD16B(NA1) cell lines

| | BG523P | LS21 | Human IgG |
|---|---|---|---|
| EMAX | 149.5 | ~−2874 | 7.456 |
| EC₅₀ (μg/mL) | 9.518 | ~155157 | 10.30 |

TABLE 5
FACS based binding of VHH to NK92mi/CD16B(NA2) cell lines

| | BG523P | LS21 | Human IgG |
|---|---|---|---|
| EMAX | 14.82 | 7.823 | 7.655 |
| EC₅₀ (μg/mL) | 28.90 | 0.1363 | 0.3139 |

### Example 2. Humanization of the Anti-Human CD16A VHH BG523P

For humanization of BG523P, human germline IgG genes were searched for sequences that share high degrees of homology to the cDNA sequences of BG523P variable regions by blasting the human immunoglobulin gene database in IMGT (http://www.imgt.org/IMGT_vquest/share/textes/index.html) and NCBI (http://www.ncbi.nlm.nih.gov/igblast/) websites. The human IGVH genes that are present in human antibody repertoires with high frequencies (Glanville 2009 PNAS 106:20216-20221) and are highly homologous to BG523P were selected as the templates for humanization.

Humanization was carried out by CDR-grafting (Methods in Molecular Biology, Vol 248: Antibody Engineering, Methods and Protocols, Humana Press) and the humanized VHH variants were engineered as VHH-Fc using an in-house developed expression vector for later binding and biophysical stability analysis, etc. In the initial round of humanization, mutations from camelid to human amino acid residues in framework regions were guided by the simulated 3D structure, and the camelid framework residues of structural importance for maintaining the canonical structures of CDRs were retained in the first versions of humanized BG523P. Among the many variants, BG524P is a preferred humanized VHH with the most retained camelid residues. Specifically, HCDR1 (SEQ ID NO: 109) and HCDR3 (SEQ ID NO: 111) of BG523P were grafted into the framework of human germline variable gene IGVH3-7 with 5 camelid framework residues (F37, R45, V78, P84, and A94 by Kabat numbering) retained, while one mutation was introduced in HCDR2 to remove a potential isomerization site. The sequences of BG524P are provided as SEQ ID NOs: 109, 114, 111, and 115-116 in Table 2.

Humanized BG523P variants were fused to the N-terminal of Fc as VT HH-Fc format using in-house developed expression vectors that contain Fc region of a human IgG1 variant (SEQ ID NO: 107), with easy adapting sub-cloning sites. Expression and preparation of humanized BG523P VHH-Fc antibodies were achieved by transfection of the constructs into ExpiCHO-s cells and by purification using a protein A column. The purified VHH-Fc antibodies were concentrated to 0.5-5 mg/mL in PBS and stored in aliquots in a −80° C. freezer.

For affinity determination, VHH-Fc antibodies were captured by an anti-human Fc surface and used in an affinity assay based on surface plasmon resonance (SPR) technology. The results of SPR-determined binding profiles of anti-CD16A VHH are summarized in Table 6. BG524P showed slightly improved binding affinities to CD16A 158V and CD16A 158F with dissociation constants at 0.08 nM and 0.08 nM, respectively, compared with those of BG523P. Meanwhile, BG524P kept the selectivity to CD16A over CD16B as characterized by SPR.

TABLE 6
Comparison of anti-CD16A VHHs binding
affinities to human CD16A by SPR

| | | |
|---|---|---|
| VHH | BG523P | BG524P |

| | | | |
|---|---|---|---|
| CD16A 158V | kₒₙ (M⁻¹s⁻¹) | 2.76E+05 | 1.35E+06 |
| | k_{off} (s⁻¹) | 1.13E−04 | 1.10E−04 |
| | K_{D} (nM) | 4.08E−10 | 8.16E−11 |
| CD16A 158F | kₒₙ (M⁻¹s⁻¹) | 3.19E+05 | 1.44E+06 |
| | k_{off} (s⁻¹) | 1.28E−04 | 1.19E−04 |
| | K_{D} (nM) | 4.02E−10 | 8.25E−11 |
| CD16B NA1 | K_{D} (nM) | 1.70E−06 | 9.42E−07 |
| CD16B NA2 | K_{D} (nM) | 4.26E−06 | 1.05E−06 |

### Using CD16A 158F Over-Expressing Cell Line NK92mii/CD16A F158 to Evaluate the Binding Activity of Anti-CD16A VHH-Fc Antibodies to Bind Native CD16A on Live Cells

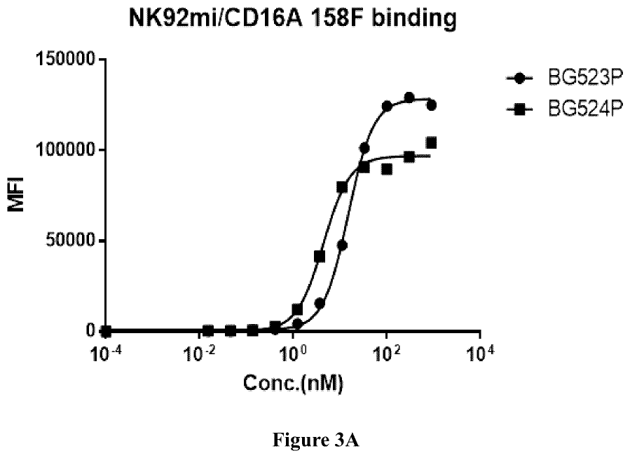

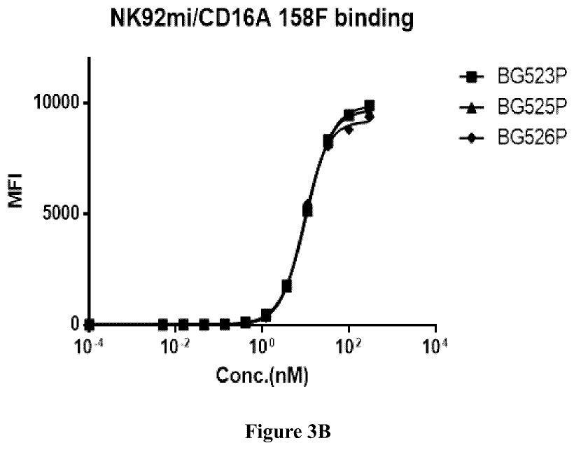

Live NK92mi/CD16A 158F cells were seeded in 96-well plates and were incubated with a series of dilutions of anti-CD16A VHH-Fc. Goat anti-human IgG was used as a second antibody to detect antibody binding to the cell surface. EC₅₀ values for dose-dependent binding to human native CD16A were determined by fitting the dose-response data to the four-parameter logistic model with GraphPad Prism. As shown in FIG. 3A and Table 7, BG524P showed improved binding affinity to native CD16A 158F but showed decreased Emax.

TABLE 7
Comparison of anti-CD16A VHHs binding
affinities to human CD16A by FACS

| VHH | BG523P | BG524P |
|---|---|---|
| NK92mi/CD16A 158F binding EC₅₀ (nM) | 15.06 | 4.42 |
| NK92mi/CD16A 158F binding Emax (MFI) | 128500 | 96964 |

To determine if the humanized BG523P kept the optimal biophysical stability of BG523P, the melting temperature (Tin) and aggregation temperature (Tagg) of BG524P were determined and compared with those of BG523P. BG524P showed both inferior Tm and Tagg compared with those of BG523P (Table 8).

Melting temperature (Tin) was determined using a high throughput MicroCal™ VP-Capillary DSC (Malvern Instruments, Northampton, MA). Thermograms for each protein (350 μL at 0.5 mg/mL) were obtained from 20° C. to 100° C. using a scan rate of 60° C./hr. Thermograms of the buffer alone were subtracted from each protein sample. Obtained results show the values for the midpoint of transition temperatures (Tm) and the calorimetric enthalpy (ΔH) of the sample.

The aggregation temperature Tagg (° C.) is representative of the colloidal stability of the samples and was obtained by monitoring the onset of aggregation by SLS266 using UNCLE™ (Unchained lab, Pleasanton, CA). Samples were loaded into Uni, and subjected to a temperature ramping from 15° C. to 95° C. The back-reflection optics cannot detect near UV light scattering by protein aggregates, and thus only non-scattered light reaches the detector. The reduction of back reflected light is therefore a direct measure for aggregation in the sample.

TABLE 8
Comparison of thermal stabilities and colloidal
stabilities of anti-CD16A VHHs

| VHH-Fc | Tm (° C.) | Tagg (° C.) |
|---|---|---|
| BG523P | 69.1 | 68.0 |
| BG524P | 59.7 | 54.5 |

BG524P was further engineered by introducing mutations in CDRs and back mutations in framework regions to improve biophysical properties, remove PTM sites, and recover binding Emax to native CD16A for therapeutic use in humans.

Taken together, the well-engineered versions of humanized monoclonal antibodies, BG525P (SEQ ID NOs: 109-111 and 117-118) and B526P (SEQ ID NOs: 109, 114, 111, and 119-120), were derived from the mutation process described above, and both retained binding affinity to CD16A, selectivity over CD16B, and optimal biophysical stability of the parental clone, as characterized in detail in Tables 9 to 11 and FIG. 3B.

TABLE 9
Comparison of anti-CD16A VHHs binding
affinities to human CD16A by SPR

| VHH | | BG523P | BG525P | BG526P |
|---|---|---|---|---|
| CD16A 158V | kₒₙ (M⁻¹s⁻¹) | 2.80E+05 | 2.55E+05 | 2.89E+05 |
| | k_{off} (s⁻¹) | 6.22E−05 | 1.03E−04 | 1.33E−04 |
| | K_{D} (nM) | 2.22E−10 | 4.04E−10 | 4.25E−10 |
| CD16A 158F | kₒₙ (M⁻¹s⁻¹) | 2.24E+05 | 2.10E+05 | 2.30E+05 |
| | k_{off} (s⁻¹) | 7.88E−05 | 1.20E−04 | 1.48E−04 |
| | K_{D} (nM) | 3.53E−10 | 5.75E−10 | 6.40E−10 |
| CD16B NA1 | K_{D} (nM) | 1.76E−06 | 2.31E−06 | 2.74E−06 |
| CD16B NA2 | K_{D} (nM) | 4.95E−06 | 5.78E−06 | 7.64E−06 |

TABLE 10
Comparison of anti-CD16A VHHs binding
affinities to human CD16A by FACS

| VHH | BG523P | BG525P | BG526P |
|---|---|---|---|
| NK92mi/CD16A 158F binding | 10.6 | 10.3 | 9.1 |
| EC₅₀ (nM) | | | |
| NK92mi/CD16A 158F binding | 9915 | 9708 | 9197 |
| EMAX(MFI) | | | |

TABLE 11
Comparison of thermal stabilities and colloidal
stabilities of anti-CD16A VHHs

| VHH-Fc | BG523P | BG525P | BG526P |
|---|---|---|---|
| Tm (° C.) | 69.1 | 69.1 | 68.1 |
| Tagg (° C.) | 68 | 68.1 | 65.2 |

### Example 3. Native CD16B Binding of Anti-CD16A VHHs

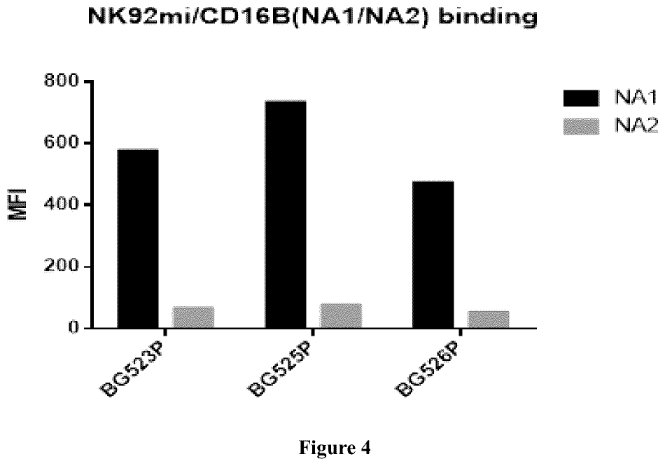

To evaluate the ability of anti-CD16A VHH to bind native CD16B on live cells, NK92mi cells were engineered to over-express human CD16B NA1 or NA2. Live NK92mi/CD16B cells were seeded in 96-well plates, and were incubated with 300 nM of anti-CD16A VHH-Fc. Goat anti-human IgG was used as a second antibody to detect anti-CD16A VHH-Fc binding to the cell surface. The binding signals of humanized VHH-Fc to CD16B were comparable with or lower than those of the parental clone as shown in FIG. 4 and Table 12, and were significantly lower than corresponding binding signals of those to CD16A (Table 10, FIGS. 3A-3B).

TABLE 12
Comparison of anti-CD16A VHHs with parental
clone on the binding to human CD16B by FACS

| | NK92mi/CD16B NA1 | NK92mi/CD16B NA2 |
|---|---|---|
| | Binding signal at | Binding signal at |
| VHH | 300 nM (MFI) | 300 nM (MFI) |
| BG523P | 577 | 65 |
| BG525P | 733 | 75 |
| BG526P | 473 | 52 |

### Example 4. Human IgG Competition on VHH Binding to Native CD16A

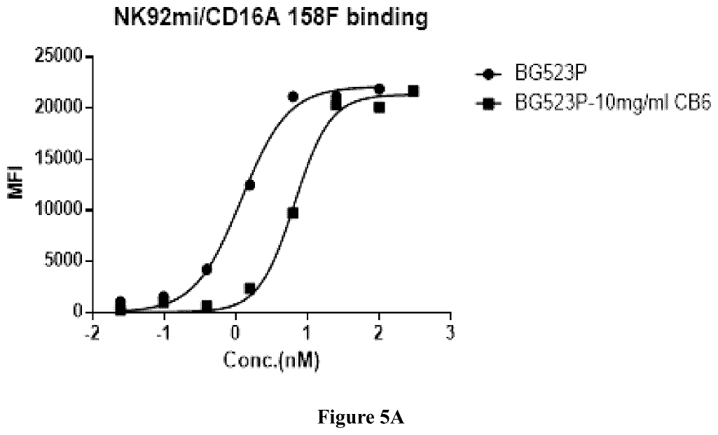

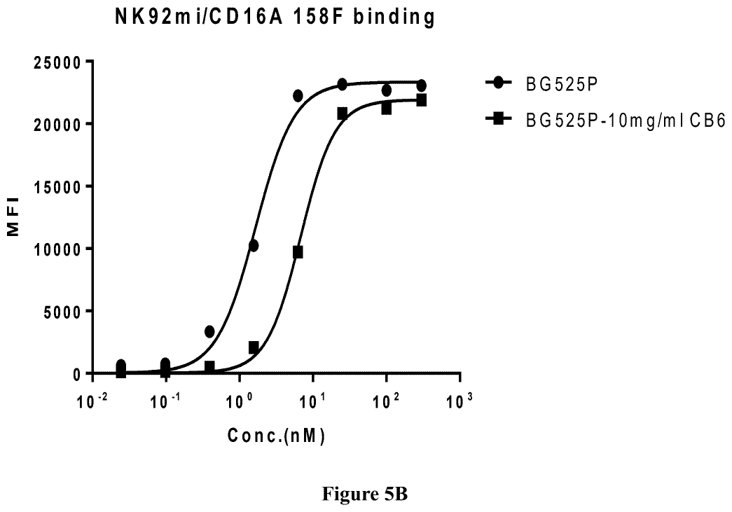

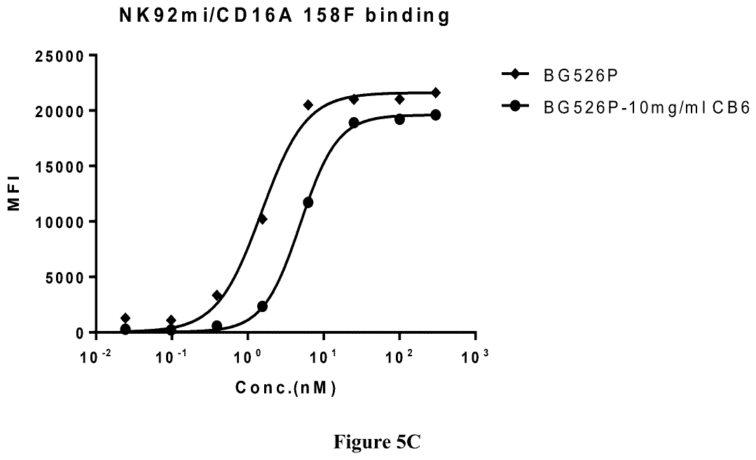

To evaluate the effect of human IgG competition on the ability of anti-CD16A VHH-Fc to bind native CD16A on live cells, a FACS-based assay was performed with or without the presence of human IgG. Live NK92mi/CD16A cells were seeded in 96-well plates, and were incubated at 37° C. with a series of dilutions of biotinylated anti-CD16A VH-1H-Fc alone or together with 10 mg/ml of a human IgG1 antibody CB6 (anti-SARS-Covid19 antibody) (SEQ ID NO: 121-122). Streptavidin-AF647 was used as a second antibody to detect biotinylated anti-CD16A VHH-Fc binding to the cell surface. EC₅₀ values for dose-dependent binding to human native CD16A were determined by fitting the dose-response data to the four-parameter logistic model with GraphPad Prism. As shown in FIGS. 5A-5C and Table 13, the binding of BG525P and BG526P to CD16A 158F were similarly affected by the presence of human IgG compared with that of parental BG523P.

TABLE 13
Human IgG competition on BG523P derived anti-CD16A
VHHs binding affinities to human CD16A 158F by FACS

| VHH | | BG523P | BG525P | BG526P |
|---|---|---|---|---|
| Without | NK92mi/CD16A 158F | 1.19 | 1.65 | 1.52 |
| human | binding EC₅₀ (nM) | | | |
| IgG1 | NK92mi/CD16A 158F | 22122 | 23336 | 21614 |
| (CB6) | binding EMAX (MFI) | | | |
| With 10 | NK92mi/CD16A 158F | 6.57 | 6.83 | 4.95 |
| mg/ml | binding EC₅₀ (nM) | | | |
| human | NK92mi/CD16A 158F | 21335 | 21914 | 19609 |
| IgG1 | binding EMAX (MFI) | | | |
| (CB6) | | | | |

### Example 5. Binding Affinity of Humanized CD16A to Cyno CD16 by SPR

For affinity determination, VHH-Fcs were captured by anti-human Fc surface and used in an affinity assay based on surface plasmon resonance (SPR) technology. The results of SPR-determined binding profiles of anti-CD16A VHH-Fc are summarized in Table 14. Humanized anti-CD16A VHH-Fcs retained cross-reactivity to cyno CD16.

TABLE 14
Binding affinities of anti-CD16A VHHs to cyno CD16 by SPR

| | Cyno CD16 | | |
|---|---|---|---|
| VHH | kₒₙ (M⁻¹s⁻¹) | k_{off} (s⁻¹) | K_{D} (nM) |
| BG523P | 2.80E+05 | 8.09E−05 | 2.89E−10 |
| BG525P | 2.67E+05 | 1.12E−04 | 4.18E−10 |
| BG526P | 3.01E+05 | 1.47E−04 | 4.90E−10 |

1. An anti-CD16A antibody or antigen-binding fragment thereof, comprising an antibody or binding fragment thereof that specifically binds to human CD16A.

2. The anti-CD16A antibody or antigen-binding fragment thereof of claim 1, wherein the antibody or antigen-binding fragment has selectivity for human CD16A over human CD16B.

3. The anti-CD16A antibody or antigen-binding fragment thereof of claim 2, wherein the antibody or antigen-binding fragment has cross-binding affinity to both human and cyno CD16A.

4. The anti-CD16A antibody or antigen-binding fragment thereof of any one of claims 1-3, wherein the antibody or antigen-binding fragment is a monoclonal antibody, a single chain antibody (scFv), a Fab fragment, a F(ab′)₂ fragment, a heavy chain antibody (HcAb), or a VHH.

5. The anti-CD16A antibody or antigen-binding fragment thereof of any one of the preceding claims, wherein the antigen-binding fragment is a VHH.

6. The anti-CD16A antibody or antigen-binding fragment thereof of any one of the preceding claims, wherein the antibody is a chimeric antibody, a humanized antibody, or a human engineered antibody.

7. The anti-CD16A antibody or antigen-binding fragment thereof of any one of the preceding claims, wherein the antibody or antigen-binding fragment comprises at least one heavy chain CDR selected from the group consisting of:
a) a heavy chain CDR1 sequence of SEQ ID NO: 109;
b) a heavy chain CDR2 sequence selected from the group consisting of SEQ ID NO: 110 and 114; and
c) a heavy chain CDR3 sequence of SEQ ID NO: 111.

8. The anti-CD16A antibody or antigen-binding fragment thereof of any one of the preceding claims, wherein the antibody comprises each of the following heavy chain CDRs:
a) a heavy chain CDR1 sequence of SEQ ID NO: 109;
b) a heavy chain CDR2 sequence selected from the group consisting of SEQ ID NO: 110 and 114; and
c) a heavy chain CDR3 sequence of SEQ ID NO: 111.

9. The anti-CD16A antibody or antigen-binding fragment thereof of any one of the preceding claims, wherein the antibody or antigen-binding fragment comprises a heavy chain variable region that comprises:
a) a HCDR1 of SEQ ID NO: 109, (b) a HCDR2 of SEQ ID NO: 110, and (c) a HCDR3 of SEQ ID NO: 111; or
b) a HCDR1 of SEQ ID NO: 109, (b) a HCDR2 of SEQ ID NO: 114, and (c) a HCDR3 of SEQ ID NO: 111.

10. The anti-CD16A antibody or antigen-binding fragment thereof of any one of the preceding claims, wherein the antibody or antigen-binding fragment comprises a heavy chain variable region (VH) comprising at least one amino acid sequence at least 90, 91, 92, 93, 94, 95, 96, 97, 98, or 99% identical to SEQ ID NO: 112, 115, 117, or 119.

11. The anti-CD16A antibody or antigen-binding fragment thereof of any one of the preceding claims, wherein the antibody or antigen-binding fragment comprises at least one amino acid sequence selected from SEQ ID NO: 112, 115, 117, and 119, and one, two, three, four, five, six, seven, eight, nine, or ten amino acids within at least one of SEQ ID NO: 112, 115, 117, and 119 have been inserted, deleted or substituted.

12. The anti-CD16A antibody or antigen-binding fragment thereof of any one of the preceding claims, wherein the antibody or antigen-binding fragment comprises at least one amino acid sequence selected from the group consisting of SEQ ID NO: 112, 115, 117, and 119.

13. A construct comprising the anti-CD16A antibody or antigen-binding fragment thereof of any one of the preceding claims.

14. The construct of claim 13, wherein the antibody or antigen-binding fragment thereof is a VHH.

15. The construct of claim 13 or 14, wherein the construct is a multi-specific antibody, a heavy chain antibody, a bivalent VHH, a biparatopic VHH, a bispecific VHH, a VHH-scFv.

16. The construct of claim 15, wherein the construct is a multi-specific antibody.

17. The construct of claim 16, wherein the multi-specific antibody is a bi-specific antibody.

18. A pharmaceutical composition comprising the anti-CD16A antibody or antigen-binding fragment thereof of any one of the preceding claims and a pharmaceutically acceptable carrier.

19. An isolated nucleic acid that encodes the anti-CD16A antibody or antigen-binding fragment thereof or construct of any one of claims 1-17.

20. A vector comprising the nucleic acid of claim 19.

21. A host cell comprising the nucleic acid of claim 19 or the vector of claim 20.

22. A process for producing an anti-CD16A antibody or antigen-binding fragment thereof comprising cultivating the host cell of claim 21 and recovering the antibody or antigen-binding fragment or construct from the culture.
